(19) United States
(12) Patent Application Publication
(10) Pub. No.: US 2025/0345426 A1
(43) Pub. Date: Nov. 13, 2025

(54) METHODS FOR MAKING AND USING THERAPEUTIC CELLS

(71) Applicant: THE REGENTS OF THE UNIVERSITY OF CALIFORNIA, Oakland, CA (US)

(72) Inventors: Yvonne Yu-Hsuan CHEN, Los Angeles, CA (US); Laurence Chaoyuen CHEN, Los Angeles, CA (US)

(73) Assignee: THE REGENTS OF THE UNIVERSITY OF CALIFORNIA, Oakland, CA (US)

(21) Appl. No.: 18/867,546

(22) PCT Filed: May 23, 2023
(86) PCT No.: PCT/US2023/023239

§ 371 Date: Nov. 20, 2024

Related U.S. Application Data
(60) Provisional application No. 63/344,942, filed on May 23, 2022.

(51) Int. Cl. A61K 40/11; A61K 31/436; A61K 31/52; A61K 31/675; A61K 35/17; A61K 38/20; A61K 40/31; A61P 35/02; C12N 5/0783; C12N 15/86; G01N 33/50

(52) U.S. Cl. CPC A61K 40/11; A61K 31/436; A61K 31/52; A61K 31/675; A61K 35/17; A61K 38/2013; A61K 38/2086; A61K 40/31; A61P 35/02; C12N 5/0636; C12N 15/86; G01N 33/5005; A61K 2239/48

(57) ABSTRACT

Aspects of the disclosure relate to a method for modifying a chimeric antigen receptor (CAR)-T cell comprising contacting the CAR-T cell ex vivo with an inhibitor, wherein the inhibitor comprises an inhibitor of PI3K, AKT, mTOR, c-MYC, SREBP, or combinations thereof. Further aspects relate to a CAR-T cell or cells made by the method of the disclosure. Also described are populations of CAR-T cells made by methods of the disclosure or derived from cells of the disclosure. The term “derived” refers to a cell that is the progeny of the cell from which it is derived. It may have further modifications or may be unmodified from the parent cell. Further aspects relate to a method for treating a subject comprising administering the cell or cells of the disclosure. Compositions comprising an inhibitor of PI3K, AKT, mTOR, c-MYC, SREBP, or combinations thereof and CAR-T cells are also disclosed.

This application claims priority of U.S. Provisional Patent Application No. 63/344,942, filed May 23, 2022, which is hereby incorporated by reference in its entirety.

The application contains a Sequence Listing in compliance with ST.26 format and is hereby incorporated by reference in its entirety. Said Sequence Listing, created on May 22, 2023 is named UCLAP0160WO.xml and is 49,152 bytes in size.

### BACKGROUND OF THE INVENTION

### I. Field of the Invention

This invention relates to the field of methods of cancer biology and cellular therapies.

### II. Background

Chimeric antigen receptor (CAR)-T cell therapy has demonstrated impressive clinical efficacy against a small subset of cancers. However, suboptimal receptor design and manufacturing processes lead to premature onset of CAR-T cell dysfunction, resulting in poor durability and weak potency of the CAR-T cell product that precludes broad applicability of CAR-T cell therapy. Antigen-independent CAR signaling, also referred to as tonic signaling or autonomous signaling, has been described as a major contributor to T-cell dysfunction. Despite this knowledge, iterative design-build-test cycles of CARs are technically challenging, expensive, and time-consuming, and this high barrier for successful CAR optimization hinders rapid translation of novel CARs into the clinic. Therefore, there is a need in the art for methods for modulation of tonic signaling

### SUMMARY

The inventors propose to overcome the challenge of CAR-T cell dysfunction and the associated barriers in CAR optimization by pre-conditioning CAR-T cells with and/or by the in vivo administration of pharmacological inhibitors targeting phosphoinositide 3-kinases (PI3K), protein kinase B (AKT), mammalian target of rapamycin (mTOR), MYC proto-oncogene (c-MYC), and sterol regulatory-element binding proteins (SREBPs) signaling pathways. Importantly, pre-conditioning of CAR-T cells prior to adoptive transfer enables researcher-defined control over CAR-T cell tonic signaling, which can lead to prevention of premature CAR-T cell dysfunction and ultimately results in enhanced CAR-T cell durability and potency. This strategy avoids the need for extensive CAR design-build-test cycles and provides a practical method to elevate CAR-T cell efficacy.

Methods relate to a method for modifying a chimeric antigen receptor (CAR)-T cell comprising contacting the CAR-T cell ex vivo with an inhibitor, wherein the inhibitor comprises an inhibitor of PI3K, AKT, mTOR, c-MYC, SREBP, or combinations thereof. Also described are CAR-T cell or cells made by the method of the disclosure. Also described are populations of CAR-T cells made by methods of the disclosure or derived from cells of the disclosure. The term “derived” refers to a cell that is the progeny of the cell from which it is derived. It may have further modifications or may be unmodified from the parent cell. The methods also provide for treating a subject comprising administering the cell or cells of the disclosure. Compositions comprising an inhibitor of PI3K, AKT, mTOR, c-MYC, SREBP, or combinations thereof and CAR-T cells are also disclosed. Also provided is method for treating a subject, the method comprising administering an inhibitor of PI3K, AKT, mTOR, c-MYC, SREBP, or combinations thereof, wherein the subject is being treated with CAR-T cell therapy. The methods also include administering an inhibitor of PI3K, AKT, mTOR, c-MYC, SREBP in combination with a CAR-T cell therapy to the subject.

The methods of the disclosure may comprise or further comprise administering an inhibitor of PI3K, AKT, mTOR, c-MYC, SREBP, or combinations thereof to a subject. The methods of the disclosure may exclude the in vivo administration of an inhibitor of PI3K, AKT, mTOR, c-MYC, SREBP, or combinations thereof to a subject. The inhibitor and CAR-T cell therapy may be administered at the same time or within 1, 2, 3, 4, 5, 6, 7, 8, 9, 10, 11, 12, 13, 14, 15, 16, 17, 18, 19, 20, 21, 22, or 23 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or 1, 2, 3, 4, 5, 6, 7, 8, 9, 10, 11, 12, 13, 14, 15, 16, 17, 18, 19, or 20 weeks of each other, or any derivable range therein. The inhibitor therapy may be administered at a time period of or of at least 1, 2, 3, 4, 5, 6, 7, 8, 9, 10, 11, 12, 13, 14, 15, 16, 17, 18, 19, 20, 21, 22, or 23 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or 1, 2, 3, 4, 5, 6, 7, 8, 9, 10, 11, 12, 13, 14, 15, 16, 17, 18, 19, or 20 weeks (or any derivable range therein) before the CAR-T cell therapy. The CAR-T cell therapy may be administered at a time period of or of at least 1, 2, 3, 4, 5, 6, 7, 8, 9, 10, 11, 12, 13, 14, 15, 16, 17, 18, 19, 20, 21, 22, or 23 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or 1, 2, 3, 4, 5, 6, 7, 8, 9, 10, 11, 12, 13, 14, 15, 16, 17, 18, 19, or 20 weeks (or any derivable range therein) before the inhibitor. The inhibitor may be administered before the CAR-T cell therapy or the inhibitor may be administered after the CAR-T cell therapy.

The method may comprise or further comprise determining the metabolic activity level of the cell. Assays for determining the metabolic activity of cells are known in the art. The metabolic activity may be determined by mass spectrometry (MS), liquid chromatography-mass spectrometry (LC-MS), metabolite extraction and analysis assays, an Agilent Seahorse XF ATP Real-Time rate assay, and direct measurement of metabolites. The cell may be one that has been determined to have a high level of metabolic activity. The cell may be one that has been determined to have a low level of metabolic activity. The methods may further comprise determining the metabolic activity level of the administered cell, of immune cells from the subject, and/or of cells from a biological sample from the subject. The subject may be one that has had the metabolic activity level determined in immune cells and/or a cells from a biological sample from the subject. The biological sample may comprise a biopsy and/or a sample of cancer cells obtained from the subject. The immune cells from the subject may have been determined to have a low level of metabolic activity in the methods of the disclosure. The immune cells from the subject may have been determined to have a high level of metabolic activity in the methods of the disclosure. The cells from the biological sample from the subject may have been determined to have a low level of metabolic activity. The cells from the biological sample from the subject may have been determined to have a high level of metabolic activity.

Methods for determining metabolic activity of cells are known in the art and can include measuring nutrient uptake and/or consumption. For example, cells can be cultured in medium with a reducing agent such as glutathione and/or high concentrations of vitamins (e.g. RPMI-1640) and serum (e.g. HI-FBS at 10%). The cell culture could then be changed to RPMI containing 10% FBS or dialyzed FBS (dFBS) supplemented with 1,2-13C-glucose. Cell culture media can be collected at time points to evaluate nutrient uptake and consumption. The cell supernatents may then be prepared for mass spectrometry by the addition of HPLC-grade methanol and by centrifugation to precipitate cell debris. Clear supernatants can be harvested and analyzed by liquid chromatography followed by mass spectrometry (LC-MS). To provide accurate estimation of nutrient uptake and consumption, partial media change can be performed at time points to avoid nutrient depletion. Methanol-treated media samples can be analyzed by reversed-phase ion-pairing liquid chromatography (Vanquish UPLC; Thermo Fisher Scientific) coupled to a high-resolution orbitrap mass spectrometer. Metabolites can be identified by comparing mass-to-charge (m/z) ratio and retention time to previously validated standards. Samples may be detected in both negative-ion mode and positive-ion mode. Negative-ion mode can be separated into two subgroups—nlo and nhi—to obtain data with m/z ratio from 60 to 200 and 200 to 2000, respectively. LC-MS data can be processed using Metabolomic Analysis and Visualization Engine (MAVEN). Labeling fractions can be corrected for the naturally occurring abundance of 13C. Concentration of metabolites in culture media can be quantified at time points by normalizing ion counts from LC-MS measurement to controls with known concentrations. Uptake and secretion rates can be calculated by subtracting sample concentration from fresh media and normalizing to viable cell count (positive values indicated secretion and negative values indicated uptake). A mole balance can be performed to account for media change from cell cultures.

A further method for determining the metabolic level of a cell or population of cells is a Agilent Seahorse XF ATP Real-Time rate assay. This assay measures and quantifies the rate of ATP production from glycolytic and mitochondrial system simultaneously using label-free technology in live cells. This assay can be performed using a commercially available kit from Agilent (Part No.: 103591-100 and 103592-100). The direct measurement of specific metabolites can also be performed using methods known in the art. For example, Promega provides for a Glucose Uptake-Glo™ Assay (Product Nos.: J1341, J1342, and J1343).

The high metabolic activity may be a level of metabolic activity of a cell that expresses a CAR with a high level of antigen-independent signaling. Examples of these include a rituximab-based CD20 CAR and/or a GD2 CAR containing a CD28 costimulatory domain. Similarly, a low metabolic activity may be a level of metabolic activity of a cell that expresses a CAR with a low level of antigen-independent signaling. An example of this includes a CD19 CAR with either CD28 or 4-1BB costimulatory domain. The measured metabolic level may be further defined as a basal level of metabolic activity that his present in the absence of antigen stimulation of the CAR.

The method may be further defined as a method for increasing antigen-independent activation of the CAR-T cells, and wherein the inhibitor comprises a c-MYC and/or SREBP inhibitor. The method ma be further defined as a method for decreasing antigen-independent activation of the CAR-T cells, and wherein the inhibitor comprises one or more of a PI3K, AKT, or mTOR inhibitor. The method may be for treating cancer in a subject having cancer.

The inhibitor may be selected from CAL-101, AZD5363, Rapamycin, MYCi975, Betulin, and combinations thereof. The inhibitor may exclude CAL-101, AZD5363, Rapamycin, MYCi975, and/or Betulin. The concentration of the inhibitor in contact with the cell may be 1 nM-1 mM. The concentration of the inhibitor in contact with the cell may be, be at least, or be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 pM, nM, or mM, or any derivable range therein.

Contacting the CAR-T cell ex vivo with the inhibitor may comprise culturing the cell in cell medium comprising the inhibitor. The concentration of the inhibitor in the cell medium may be 1 nm-1 mM. The concentration of the inhibitor in contact with the cell may be 1 nM-1 mM. The concentration of the inhibitor in the cell medium may be, be at least, or be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 pM, nM, or mM, or any derivable range therein.

The inhibitor may comprise CAL-101 at a concentration of 1 μM. The inhibitor may comprise AZD5363 at a concentration of 1 μM. The inhibitor may comprise Rapamycin at a concentration of 100 nM. The inhibitor may comprise MYCi975 at a concentration of 2 μM. The inhibitor may comprise Betulin at a concentration of 10 μM. The methods or the subjects of the disclosure may exclude the ex vivo administration of an inhibitor of PI3K, AKT, mTOR, c-MYC, SREBP, or combinations thereof to a cell.

The cell medium may comprise or the methods may comprise contacting the cell with cytokine. The cell medium or the methods may exclude contacting the cell with cytokine. The cytokines may comprise or consist of IL-2 and/or IL-15. The concentration of IL-2 may be 50 U/mL and/or the concentration of IL-15 may be 1 ng/mL. The concentration of IL-2 may be 50 U/mL and/or the concentration of IL-15 may be 0.5 ng/ml. The IL-2 may be in contact with the cells or in the cell medium at a concentration of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 IU/mL, or any derivable range therein. The IL-15 may be in contact with the cells or in the cell medium at a concentration of at least, at most, or exactly 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 ng/mL, or any derivable range therein.

The CAR-T cell may comprise a stimulated CAR-T cells. The CAR-T cell may be one that has been stimulated with CD3 and/or CD28 antibodies or antigen-binding fragments. The medium or methods may exclude CD3 and/or CD28 activating molecules. The CAR-T cell may be one that has been stimulated with CD3/CD28 Dynabeads. The CAR-T cell may be one that has been stimulated with TransAct™. The cells may be stimulated with MACS® GMP T cell TransAct™. MACS® GMP T cell TransAct™ is available commercially through, for example, Miltenyi Biotec. Stimulation with MACS® GMP T cell TransAct™. MACS® GMP T cell TransAct™ may be excluded in the methods described herein. The product format is described as a polymeric nanomatrix conjugated to recombinant humanized CD3 and CD28 agonist supplied in phosphate buffered-saline (PBS), containing 0.03% poloxamer 188 and 5 g/L recombinant human serum albumin, pH 7.3-7.9. The capacity of the reagent is sufficient to activate and expand up to 2×10⁸ enriched T cells or up to 4×10⁸ PBMC in a maximal volume of 70 mL, when used at recommended titer of 1:17.5. The transactivating composition may be used at a titer of 1:25-1:40. The composition may be diluted to 1:35. The dilution or titer of the transactivating composition may be, be at least, or be at most 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, or 1:50.

The cells or population of cells of the disclosure may be maintained in a cell culture medium throughout the methods of the claims. At any step of the process, the cell culture medium may be serum-free. The process may be one that maintains the cells in serum-free medium throughout all of the steps. The methods may exclude contacting the cells with serum. The method may comprise or further comprise evaluating the population of cells comprising T cells for the CD14 and/or CD25 cell marker. The percentage of CD14+ and CD25+ cells may be determined or evaluated to be greater than or equal to 5%. The percentage of CD14+ and CD25+ cells evaluated or determined may be less than or equal to about 5%. The percentage of CD3+ cells evaluated or determined may be more than or equal to about 15%. The percentage of CD14+, CD3+, and/or CD25+ cells may be determined or evaluated to be exactly, greater than, or less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any derivable range therein. The method may comprise or further comprise depleting the population of cells of CD14+ and/or CD25+ cells; the depleted population of cells may be reduced by about, at least about, or at most about 60, 65, 70, 75, 80, 85, 90, 95, 96, 97, 98, 99 or 100% (or any range derivable therein) with respect to cells having one or both of CD14 and CD25. Depleting the population of cells of CD14+ and/or CD25+ cells may comprise or further comprise contacting the cells with anti-CD14 and/or anti-CD25 microbeads. The cells may be ones that are not depleted for CD14+ or CD25+ cells.

The CAR-T cell may be or may be derived from a CD8+ T cell. The CAR-T cell may be a CD3+ T cell or may be derived from a CD3+ T cell. The CAR-T cell may be a naïve and/or memory T cell or may be derived from a naïve and/or memory T cell. The naïve or memory T cell may comprise a CD14−/CD25−/CD62+ T cell. The naïve or memory T cell may comprise a CD62+ T cell. The naïve and/or memory T cell may be one that is not derived from a population of cells that have been depleted for CD14 and CD25-positive cells. CAR-T cells that are or that are derived from a CD8+ T cell, a CD3+ T cell, a naïve T cell, a memory T cell, and/or a CD14−/CD25−/CD62+ T cell may be excluded in the methods, compositions, and cells of the disclosure.

The CAR-T cell or cells may be contacted with the inhibitor for a period of time of at least 48 hours. The period of time may be, be at least, or be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 hours, or any derivable range therein.

The method may be further defined as a method for increasing antigen-independent activation of the CAR-T cells, and wherein the inhibitor comprises a c-MYC and/or SREBP inhibitor. The method may be further defined as a method for decreasing antigen-independent activation of the CAR-T cells, and wherein the inhibitor comprises one or more of a PI3K, AKT, or mTOR inhibitor.

The population of cells comprising T cells may comprise cells that have been isolated from a patient, such as a human patient, by leukapheresis. Cells isolated by leukapheresis may be excluded in the methods, compositions, and cells of the disclosure. Leukapheresis is a laboratory procedure in which white blood cells are separated from a sample of blood. It is a specific type of apheresis, the more general term for separating out one particular constituent of blood and returning the remainder to the circulation. Leukapheresis may be performed to decrease a very high white blood cell count, to obtain blood cells from a patient (autologous) or donor (allogeneic) for later transplant into the patient, or to obtain cells for research purposes. Leukapheresis may be performed to obtain the patient's own blood cells for a later transplant. The population of cells comprising T cells may be human cells. The population of cells comprising T cells may be further defined as primary cells.

The CAR T cells may comprise a CAR that is known to have antigen-independent signaling or a CAR that is known to lack antigen-independent signaling. The CAR may be one that has high antigen-independent signaling or low antigen-independent signaling. The CAR may be a comprises a CD19, CD20, bispecific, monospecific, IL13Ra2, BCMA, GD2, EGFRVIII, TGF-β, or CS1 CAR. The CAR may exclude a CD19, CD20, bispecific, monospecific, IL13Ra2, BCMA, GD2, EGFRVIII, TGF-β, or CS1 CAR.

The CAR may be a CD20 CAR. The CD20 CAR may comprise a Rituximab-based scFv. The CD20 CAR may comprise a scFv comprising a variable heavy (VH) and variable light (VL) region, wherein the VH comprises complementarity determining regions (CDRS) HCDR1, HCDR2, and HCDR3 and the VL comprises LCDR1, LCDR2, and LCDR3; wherein HCDR1, HCDR2, HCDR3, LCDR1, LCDR2, and LCDR comprise the amino acid sequences of SEQ ID NOS: 19-24, respectively, or an amino acid sequence with at least 80% sequence identity to SEQ ID NOS: 19-24, respectively. The CD20 CAR may comprise a scFv comprising a variable heavy (VH) and variable light (VL) region, wherein the VH comprises complementarity determining regions (CDRS) HCDR1, HCDR2, and HCDR3 and the VL comprises LCDR1, LCDR2, and LCDR3; wherein HCDR1, HCDR2, HCDR3, LCDR1, LCDR2, and LCDR comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NOS: 19-24, respectively. The VL and VH may comprise the amino acid sequences of SEQ ID NOS: 3-4, respectively, or an amino acid sequence with at least 80% sequence identity to SEQ ID NOS: 3-4, respectively. The VL and VH may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any range derivable therein) to SEQ ID NOS: 3-4, respectively.

The CAR may comprise a Leu16 scFv. The CD20 CAR may comprise a scFv comprising a variable heavy (VH) and variable light (VL) region, wherein the VH comprises complementarity determining regions (CDRS) HCDR1, HCDR2, and HCDR3 and the VL comprises LCDR1, LCDR2, and LCDR3; wherein HCDR1, HCDR2, HCDR3, LCDR1, LCDR2, and LCDR comprise the amino acid sequences of SEQ ID NOS: 5-10, respectively, or an amino acid sequence with at least 80% sequence identity to SEQ ID NOS: 5-10, respectively. The VL and VH may comprise the amino acid sequences of SEQ ID NOS: 1-2, respectively, or an amino acid sequence with at least 80% sequence identity to SEQ ID NOS: 1-2, respectively. The VL and VH may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any range derivable therein) to SEQ ID NOS: 1-2, respectively.

The cells may be transduced with a viral vector. The viral vector may be a lentiviral-based virus comprising a nucleic acid encoding the CAR. The virus may be packaged in a packaging cell. The virus may be packaged in HEK-293T cells. Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). The nucleic acid may comprise an epHIV7 vector backbone. Other suitable viral vectors include, for example, pRSV-Rev, pMDLg/pRRE, psPAX2, pCMV delta R8.2, pMD2.G, pCMV-VSV-G, pCMV-dR8.2 dvpr, pCI-VSVG, pCPRDEnv, pLTR-RD114A, pLenti-III (Applied Biological Materials; cat #LV587); 87 pLentiCRISPR v.1 (Addgene; cat #52963); 88 p156RRLsinppt (Addgene; cat #42795); 89 pFUGW (Addgene; cat #14883); 90 pFUG (Addgene; cat #14882); 90 pHAGE (Addgene; cat #46793); 91 pHRsin (Addgene; cat #12265); 92 pLenti (AMP) (Addgene; cat #61422); 93 pLK0.1 (Addgene; cat #10878); 94 pLL3.7 m (Addgene; cat #89362); 95 Puro.cre (Addgene; cat #17408); 96 pRSIEG (Cellecta; SVSHU6EG-L); pLenti7.3 (Thermo Fisher Scientific; cat #V53406); pLenti (OriGene; cat #PS100109); pSF_Lenti (Sigma; cat #OGS269); pLV-GFPSpark (Sinobiological; cat #LVCV-01); pLVX (Takara; cat #632164); pALD-Lenti (Aldevron), pLenti (Vigene; cat #P100020), pLenti CMV (Addgene), and pLV. The viral vector may exclude epHIV7, pRSV-Rev, pMDLg/pRRE, psPAX2, pCMV delta R8.2, pMD2.G, pCMV-VSV-G, pCMV-dR8.2 dvpr, pCI-VSVG, pCPRDEnv, pLTR-RD114A, pLenti-III (Applied Biological Materials; cat #LV587); 87 pLentiCRISPR v.1 (Addgene; cat #52963); 88 p156RRLsinppt (Addgene; cat #42795); 89 pFUGW (Addgene; cat #14883); 90 pFUG (Addgene; cat #14882); 90 pHAGE (Addgene; cat #46793); 91 pHRsin (Addgene; cat #12265); 92 pLenti (AMP) (Addgene; cat #61422); 93 pLK0.1 (Addgene; cat #10878); 94 pLL3.7 m (Addgene; cat #89362); 95 Puro.cre (Addgene; cat #17408); 96 pRSIEG (Cellecta; SVSHU6EG-L); pLenti7.3 (Thermo Fisher Scientific; cat #V53406); pLenti (OriGene; cat #PS100109); pSF_Lenti (Sigma; cat #OGS269); pLV-GFPSpark (Sinobiological; cat #LVCV-01); pLVX (Takara; cat #632164); pALD-Lenti (Aldevron), pLenti (Vigene; cat #P100020), pLenti CMV (Addgene), and/or pLV.

In the transduced cells, the MOI is at least, at most, or exactly 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10, or any derivable range therein. The cells may be transduced at a concentration of 1×10⁶ cells/mL. The transduced cells may be at a concentration of at least, at most, or exactly 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, or 1×10¹⁴ cells/mL (or any derivable range therein). The cells may be expanded after the transduction. The cells may be expanded 1.5-25 folds after transduction. The cells may be expanded to, to at least, or to at most 2, 3, 4, 5, 6, 7, 8, 9, 10, 11, 12, 13, 14, 15, 16, 17, 18, 19, or 20 fold (or any derivable range therein) after transduction. Expansion, splitting, or passage of the cells may be excluded in the methods or compositions of the disclosure.

The nucleic acid encoding the CAR may also be transferred into the cell by non-viral methods, such as gene editing and transfection procedures described herein. The method may comprise or further comprise cryopreservation of the cell before or after contacting the cell with the inhibitor.

The methods may comprise or further comprise cryopreserving the cells. The cells may be cryopreserved at a concentration of 1×10⁶ cells/mL-15×10⁶ cells/mL. The cells may be cryopreserved at a concentration of, of at least, or of at most 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, or 1×10¹⁴ cells/mL (or any derivable range therein). The cells may be cryopreserved at a time period of less than 17 days after transduction. The cells may be cryopreserved at a time period of exactly, or of less than 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days (or any derivable range therein) after transduction. The cells may be filtered prior to cryopreservation. Cryopreservation may also be excluded in the methods of the disclosure.

The transduced cells or transduced cell populations may have an average of 1-3 copies of the nucleic acid encoding the CAR per cell. The cells or cell populations may have an average of, of at least, or of at most 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8 (or any derivable range therein) copies of the nucleic acid encoding the CAR per transduced cell.

The cells or cell populations may comprise at least 70% viable cells after thawing. The cells or cell populations comprise or comprise at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% viable cells (or any derivable range therein) after thawing.

At least 10% of the cells in the cell populations may be CD3+CAR+ cells. At least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) of the cells in the cell populations may be CD3+CAR+ cells. The population may comprise a mixture of CD4+ and CD8+ T cells, such as CD4+ single positive and CD8+ single positive T cells. The ratio of CD8+ cells to CD4+ cells may be about 3:1. The ratio of CD8+ cells to CD4+ cells may be, may be at least, or may be at most 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20 to 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20 (or any derivable range therein).

The population of cells may comprise at least 5% CD4+ cells. The population of cells may comprise, comprise at least, or comprise at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% CD4+ cells. The population of cells may comprise at least 15% CD8+ cells. The population of cells may comprise, comprise at least, or comprise at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% CD8+ cells. The ratio of CD4+ to CD8+ in the cells may not be significantly changed from a control, wherein a control comprises the ratio of CD4+ to CD8+ in an untransduced sample from the patient.

1×10⁶-2×10⁸ cells may be administered. The amount of cells administered to a subject may be, may be at least, or may be at most 1×10², 2×10², 3×10², 4×10², 5×10², 6×10², 7×10², 8×10², 9×10², 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, or 1×10¹⁴ cells (or any derivable range therein). The cells may be determined to be or evaluated as positive for expression of the CAR. The cells may be autologous cells. The cells may be allogeneic.

The subject may be one that is being treated with an additional therapy. The method may further comprise administration of an additional therapy. The subject may exclude one that is being treated with an additional therapy. The method may exclude administration of an additional therapy. The additional therapy may include a therapy described herein. The additional therapy may be a chemotherapy. The additional therapy may comprise lymphodepletion. The additional therapy may include fludarabine and/or cyclophosphamide. The chemotherapy may comprise both fludarabine and cyclophosphamide. The additional therapy may be given prior to administration of the cells. The additional therapy may be given after administration of the cells. The additional therapy may be given to the subject at a time period of five days prior to administration of the cells. The additional therapy may be given to the subject at a time period of, of at least, or of at most 1, 2, 3, 4, 5, 6, 7, 8, 9, 10, 11, or 12 (or any derivable range therein) days prior to or after administration of the cells. The additional therapy may be an additional therapy described herein, such as an immunotherapy, inhibition of co-stimulatory molecules, dendritic cell therapy, CAR-T cell therapy, adoptive T-cell therapy, chemotherapy, radiotherapy, or surgery.

The subject may be administered 30 mg/m²/day for 30 min of fludarabine for three days. The subject may be administered 500 mg/m²/day for 60 min of cyclophosphamide for three days. The methods may exclude administration of fludarabine and/or cyclophosphamide. The subject may be administered, administered at least, or administered at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, (or any derivable range therein) mg/m²/day of fludarabine for 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 minutes (or any derivable range therein) for a time period of, of at least, or of at most 1, 2, 3, 4, 5, 6, 7, 8, 9, or 10 days (or any derivable range therein). The subject may be administered, administered at least, or administered at most 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000, (or any derivable range therein) mg/m²/day of cyclophosphamide for 10, 15, 20, 25, 30, 35, 40, 45, 50, 55, 60, 65, 70, 75, 80, 85, 90, 95, 100, 105, 110, 115, 120, 125, 130, 135, 140, 145, or 150 minutes (or any derivable range therein) for a time period of, of at least, or of at most 1, 2, 3, 4, 5, 6, 7, 8, 9, or 10 days (or any derivable range therein).

The cancer amenable to treatment by the methods of the disclosure may be a cancer described herein. The cancer amendable to treatment by the methods of the disclosure may exclude a cancer described herein. The cancer may be lung cancer, prostate cancer, ovarian cancer, testicular cancer, brain cancer, neuroblastoma, sarcoma, osteocarcoma, skin cancer, melanoma, colon cancer, rectal cancer, gastric cancer, esophageal cancer, tracheal cancer, head & neck cancer, pancreatic cancer, liver cancer, breast cancer, ovarian cancer, glioblastoma, glioma, a B-cell malignancy, melanoma, leukemia, sarcomas of bone or soft tissue, cervical cancer, and vulvar cancer. The cancer may comprise a B-cell malignancy. The B-cell malignancy may be relapsed/refractory B-cell malignancy. The subject may be one that has previously been treated for the B-cell malignancy. The subject may be one that has previously been treated with at least 1, at least 2, or at least 3 lines of therapy. The subject may be one that has previously been treated with or with at least 1, 2, 3, 4, 5, or 6 lines of therapy. The previous treatment may comprise Bendamustine, Rituximab, Acalabrutinib, Umbralisib, Ublituximab, Lenalidomide, cyclophosphamide, doxorubicin, vincristine, prednisone, R-CHOP (combination of rituximab, cyclophosphamide, doxorubicin, vincristine, and prednisone), ifosfamide, carboplatin, etoposide, R-ICE (combination of rituximab, ifosfamide, carboplatin, and etoposide), R-EPOCH (combination of rituximab, etoposide phosphate, prednisone, vincristine sulfate, cyclophosphamide, and doxorubicin hydrochloride), ROR1-Targeting Antibody-Drug Conjugate, anti-CD19 bispecific T cell engager; gemcitabine, oxaliplatin, dexamethasone, autologous stem cell transplantation, or combinations thereof. The previous treatment may include the combination of bendamustine and rituximab. The previous treatment may comprise the combination of umbralisib and ublituximab, The previous treatment may comprise lenalidomide and rituximab. The previous treatment may comprise rituximab, gemcitabine, and oxaliplatin. The previous treatment may comprise rituximab and dexamethasone. The previous treatment may comprise rituximab, cyclophosphamide, and etoposide. The previous treatment may comprise the combination of rituximab, gemcitabine, dexamethasone, and carboplatin. The subject may be one that has not previously been treated for the B-cell malignancy. The therapy may be an additional therapy described herein, such as an immunotherapy, inhibition of co-stimulatory molecules, dendritic cell therapy, CAR-T cell therapy, adoptive T-cell therapy, chemotherapy, radiotherapy, or surgery.

The B-cell malignancy may be a lymphoma or leukemia. The B-cell malignancy may comprise non-Hodgkin B-cell lymphoma. The non-Hodgkin B-cell lymphoma may be further classified as indolent non-Hodgkin lymphomas, follicular lymphoma, lymphoplasmacytic lymphoma, marginal zone lymphoma, nodal marginal zone lymphoma, gastric mucosa-associated lymphoid tissue (MALT) lymphoma, extragastric MALT lymphoma, mediterranean abdominal lymphoma, splenic marginal zone lymphoma, primary cutaneous anaplastic large cell lymphoma, diffuse large B-cell lymphoma, primary mediastinal B-cell lymphoma, follicular large cell lymphoma, anaplastic large cell lymphoma, cutaneous anaplastic large cell lymphoma, systemic anaplastic large cell lymphoma, extranodal NK-/T-cell lymphoma, lymphomatoid granulomatosis, angioimmunoblastic T-cell lymphoma, peripheral T-cell lymphoma, hepatosplenic T-cell lymphoma, subcutaneous panniculitis-like T-cell lymphoma, enteropathy-type intestinal T-cell lymphoma, intravascular large B-cell lymphoma, Burkitt lymphoma, lymphoblastic lymphoma, adult T-cell leukemia/lymphoma, mantle cell lymphoma, posttransplantation lymphoproliferative disorder, true histiocytic lymphoma, primary effusion lymphoma, or plasmablastic lymphoma. The B-cell malignancy may comprise leukemia, and wherein the leukemia is further classified as chronic lymphocytic leukemia, small-lymphocytic leukemia, acute lymphocytic leukemia, acute myeloid leukemia, chronic myeloid leukemia, chronic myelomonocytic leukemia, pediatric leukemia, precursor B acute lymphoblastic leukemia, precursor T acute lymphoblastic leukemia, Burkitt's leukemia, acute biphenotypic leukemia, B-cell prolymphocytic leukemia, acute promyelocytic leukemia, acute myeloblastic leukemia, acute megakaryoblastic leukemia, hairy cell leukemia, T-cell prolymphocytic leukemia, large granular lymphocytic leukemia, adult T-cell leukemia, or clonal eosinophilias.

The cancer may also comprise glioma. The glioma may be further defined as glioblastoma, malignant glioma, diffuse midline glioma, or diffuse intrinsic pontine glioma.

The method may further comprise further purifying the CAR-expressing cells based on inclusion or exclusion of other cell markers that include, for example, CD4, CD8, CD45RA, CD45RO, CCR7/CD197, CD62L, CD27, CD28, and CD1a; or, CD7, CD25, CD45, CD45RA, CD127, and CD200R. The method may exclude purifying the CAR-expressing cells based on inclusion or exclusion of other cell markers that include, for example, CD4, CD8, CD45RA, CD45RO, CCR7/CD197, CD62L, CD27, CD28, and CD1a; or, CD7, CD25, CD45, CD45RA, CD127, and CD200R.

The compositions and methods described herein may be modified so that the method is for preparing a T cell with a certain phenotype. The methods may be for preparing a T cell with the phenotype: CD4⁺CD8⁻ T cells, CD4⁻CD8⁺ T cells, CD34⁺CD7⁺CD1a⁺ cells, CD3+ TCRab+, CD3+ TCRgd+, CD3+ TCRab+CD4+CD8−, CD3+ TCRab+CD8+CD4−, CD3+ TCRab+CD4+CD8−CD45RO−CD45RA+, CD3+ TCRab+CD8+CD4−CD45RO−CD45RA+, CD3+ TCRab+CD4+CD8−CD45RO−CD45RA+CCR7+, CD3+ TCRab+CD8+CD4−CD45RO−CD45RA+CCR7+, CD3+ TCRab+CD4+CD8−CD45RO−CD45RA+CD27+, CD3+ TCRab+CD8+CD4−CD45RO−CD45RA+CD27+, CD34⁺CD7⁺CD1a⁺ cells, CD34+CD5+CD7+, CD34+CD5+CD7−, natural killer T cells, regulatory T cells, antigen-specific T cells, intraepithelial lymphocyte T cells, or cells that are CD45+, CD11b+, CD11b−, CD15+, CD15−, CD24+, CD24−, CD114+, CD114−, CD182+, CD182−, CD4+, CD4−, CD14+, CD14−, CD11a+, CD11a−, CD91+, CD91−, CD16+, CD16−, CD3+, CD3−, CD25+, CD25−, Foxp3+, Fox3p−, CD8+, CD8−, CD19+, CD19−, CD20+, CD20−, CD24+, CD24, CD38+, CD38−, CD22+, CD22−, CD61+, CD61−, CD16+, CD16−, CD56+, CD56−, CD31+, CD31−, CD30+, CD30−, CD38+, and/or CD38− or combinations thereof.

The methods may exclude the use of or preparing a T cell with the phenotype: CD4⁺CD8⁻ T cells, CD4⁻CD8⁺ T cells, CD34⁺ CD7⁺ CD1a⁺ cells, CD3+ TCRab+, CD3+TCRgd+, CD3+ TCRab+CD4+CD8−, CD3+ TCRab+CD8+CD4−, CD3+ TCRab+CD4+CD8−CD45RO−CD45RA+, CD3+ TCRab+CD8+CD4−CD45RO−CD45RA+, CD3+TCRab+CD4+CD8−CD45RO−CD45RA+CCR7+, CD3+ TCRab+CD8+CD4−CD45RO−CD45RA+CCR7+, CD3+ TCRab+CD4+CD8−CD45RO−CD45RA+CD27+, CD3+TCRab+CD8+CD4−CD45RO−CD45RA+CD27+, CD34⁺ CD7⁺ CD1a⁺ cells, CD34+CD5+CD7+, CD34+CD5+CD7−, natural killer T cells, regulatory T cells, antigen-specific T cells, intraepithelial lymphocyte T cells, or cells that are CD45+, CD11b+, CD11b−, CD15+, CD15−, CD24+, CD24−, CD114+, CD114−, CD182+, CD182−, CD4+, CD4−, CD14+, CD14−, CD11a+, CD11a−, CD91+, CD91−, CD16+, CD16−, CD3+, CD3−, CD25+, CD25−, Foxp3+, Fox3p−, CD8+, CD8−, CD19+, CD19−, CD20+, CD20−, CD24+, CD24, CD38+, CD38−, CD22+, CD22−, CD61+, CD61−, CD16+, CD16−, CD56+, CD56−, CD31+, CD31−, CD30+, CD30−, CD38+, and/or CD38− or combinations thereof.

The cells may be further defined as having the following phenotype: CD4⁺CD8⁺ T cells, CD4⁻CD8⁺ T cells, CD34⁺ CD7⁺ CD1a⁺ cells, CD3+ TCRab+, CD3+ TCRgd+, CD3+TCRab+CD4+CD8−, CD3+ TCRab+CD8+CD4−, CD3+ TCRab+CD4+CD8−CD45RO−CD45RA+, CD3+ TCRab+CD8+CD4−CD45RO−CD45RA+, CD3+ TCRab+CD4+CD8−CD45RO−CD45RA+CCR7+, CD3+ TCRab+CD8+CD4−CD45RO−CD45RA+CCR7+, CD3+ TCRab+CD4+CD8−CD45RO−CD45RA+CD27+, CD3+ TCRab+CD8+CD4−CD45RO−CD45RA+CD27+, CD34⁺ CD7⁺ CD1a⁺ cells, CD34+CD5+CD7+, CD34+CD5+CD7−, natural killer T cells, regulatory T cells, antigen-specific T cells, intraepithelial lymphocyte T cells, or cells that are CD45+, CD11b+, CD11b−, CD15+, CD15−, CD24+, CD24−, CD114+, CD114−, CD182+, CD182−, CD4+, CD4−, CD14+, CD14−, CD11a+, CD11a−, CD91+, CD91−, CD16+, CD16−, CD3+, CD3−, CD25+, CD25−, Foxp3+, Fox3p−, CD8+, CD8−, CD19+, CD19−, CD20+, CD20−, CD24+, CD24, CD38+, CD38−, CD22+, CD22−, CD61+, CD61−, CD16+, CD16−, CD56+, CD56−, CD31+, CD31−, CD30+, CD30−, CD38+, and/or CD38− or combinations thereof.

Cells of the following phenotypes may be excluded from the methods, cells, and compositions of the disclosure: CD4⁺CD8⁻ T cells, CD4⁻CD8⁺ T cells, CD34⁺ CD7⁺ CD1a⁺ cells, CD3+ TCRab+, CD3+ TCRgd+, CD3+ TCRab+CD4+CD8−, CD3+ TCRab+CD8+CD4−, CD3+ TCRab+CD4+CD8−CD45RO−CD45RA+, CD3+ TCRab+CD8+CD4−CD45RO−CD45RA+, CD3+ TCRab+CD4+CD8−CD45RO−CD45RA+CCR7+, CD3+TCRab+CD8+CD4−CD45RO−CD45RA+CCR7+, CD3+ TCRab+CD4+CD8−CD45RO−CD45RA+CD27+, CD3+ TCRab+CD8+CD4−CD45RO−CD45RA+CD27+, CD34⁺ CD7⁺ CD1a⁺ cells, CD34+CD5+CD7+, CD34+CD5+CD7−, natural killer T cells, regulatory T cells, antigen-specific T cells, intraepithelial lymphocyte T cells, or cells that are CD45+, CD11b+, CD11b−, CD15+, CD15−, CD24+, CD24−, CD114+, CD114−, CD182+, CD182−, CD4+, CD4−, CD14+, CD14−, CD11a+, CD11a−, CD91+, CD91−, CD16+, CD16−, CD3+, CD3−, CD25+, CD25−, Foxp3+, Fox3p−, CD8+, CD8−, CD19+, CD19−, CD20+, CD20−, CD24+, CD24, CD38+, CD38−, CD22+, CD22−, CD61+, CD61−, CD16+, CD16−, CD56+, CD56−, CD31+, CD31−, CD30+, CD30−, CD38+, and/or CD38− or combinations thereof.

The subject may be any animal, in particular a mouse, non-human primate, or human. The subject may have been determined to have or be at risk for cancer.

The cell population or composition of cells may comprise a ratio of at least or at most 1:0.1, 1:0.2, 1:0.3, 1:0.4, 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9, 1:10, 1:10.5, 1:11, 1:11.5, 1:12, 1:12.5, 1:13, 1:13.5, 1:14, 1:14.5, 1:15, 1:20, 1:25, 1:30, 1:35, 1:40, 1:45, or 1:50 (or any range derivable therein) of live cells:cells having a phenotype and/or cell marker described herein or ratio of cells having a phenotype and/or cell marker described herein:live cells.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-G. Pharmacological inhibition of PI3K/AKT pathway modulates antigen-independent CAR-T cell activation. (A) Table of pharmacological inhibitors used during ex vivo expansion of rituximab CAR-T cells (left). Simplified schematic of the PI3K/AKT signaling axis where black arrows represent activation (right). (B) Activation marker expression by CAR⁺ T cells in the absence of antigen stimulation after 5 days of pharmacological modulation. Data bars indicate the means of technical triplicates±1 S.D. The bars in FIG. 1B represent data from, untreated, vehicle, CAL-101, AZD5363, Rapamycin, MYCi975, and Betulin, respectively, from left to right. (C) TNF-α production CAR⁺ T cells in the absence of antigen stimulation as quantified by intracellular flow cytometry. Data bars indicate the means of technical triplicates±1 S.D. The bars in FIG. 1B and FIG. 1C represent data from, untreated, vehicle, CAL-101, AZD5363, Rapamycin, MYCi975, and Betulin, respectively, from left to right. (D) Proliferation of CAR⁺ T cells stained with CellTrace Violet (CTV) dye was assayed after a 4-day culture in the absence of target cells or exogenous cytokines. Data shown in the histogram correspond to one of technical triplicates shown in the bar graph. The bars FIG. 1D represent data from, EGFRt, untreated, vehicle, CAL-101, AZD5363, Rapamycin, MYCi975, and Betulin, respectively, from left to right. (E) % distribution of T-cell subtypes in CAR⁺ T cells after 5 days of pharmacological modulation. Data bars indicate the means of technical triplicates±1 S.D. (F) Rituximab CAR-T cell fold expansion after 6 days of inhibitor culture was tracked. T-cell fold expansion was normalized to the untreated control with each data point representing biological replicates collected from healthy donors. The bars FIG. 1F represent data from, EGFRt, vehicle, CAL-101, AZD5363, Rapamycin, MYCi975, and Betulin, respectively, from left to right. (G) A 24-hr lysis assay of CD20 CAR-T cells against Raji target cells at three effector-to-target (E:T) ratios. Data bars indicate the means of technical triplicates±1 S.D. Percent lysis was normalized to cell counts in target-only wells. In the line graph, the line that shows the least amount of % lysis represents data for EGFRt, while the lines represented by data for vehicle, CAL-101, AZD5363, Rapamycin, MYCi975, and Betulin are clustered together. In (B)-(F), results are representative two independent experiments using cells from of two heathy donors. Statistical significance in panels B, C, and D was determined by two-tailed Student's t test with Sidak correction for multiple comparisons. * p<0.05, ** p<0.01, *** p<0.001, n.s. not statistically significant.

FIG. 2A-C. Modulation of antigen-independent rituximab CAR-T cell activation leads to divergent anti-tumor efficacy in vivo. NSG mice were injected intravenously with 0.5×10⁶ ffLuc-expressing Raji cells followed by two doses of 1.35×10⁶ CAR-T cells at 7 and 12 days post tumor injection. (A) Tumor signal (in photons/sec/cm²/sr) in individual animals as quantified by bioluminescence imaging. Table of statistical differences in tumor signal compared to EGFRt-treated animals 1 week, 2 weeks, or 1 month after the 2^{nd} T-cell injection. Statistical significance was determined by Mann-Whitney U test with Holm-Sidak correction for multiple comparisons. * p<0.05, n.s. not statistically significant. (B) Kaplan-Meier survival curve (top) and table of median survival period in days (bottom). (C) Frequency of human CD45+EGFRt+ cell in peripheral blood collected from mice on day 8 after first dose of T-cell infusion. Statistical significance was determined by two-tailed Student's t test with Sidak correction for multiple comparisons. * p<0.05, ** p<0.01.

FIG. 3A-G. Pharmacological modulation of Leu16 CAR-T cells modestly dampens tonic signaling while enhancing anti-tumor efficacy. (A) Pictorial representation of the correlation between tonic signaling-modulated rituximab CAR-T cells and in vivo anti-tumor efficacy. (B) Pictorial representation of pharmacological modulation strategy aimed to either increase or decrease Leu16 CAR tonic signaling; c-MYC (MYCi975) and SREBP (betulin) inhibition increased tonic signaling whereas mTORC1 (rapamycin) inhibition decreased tonic signaling in rituximab CAR-T cells. (C) % distribution of T-cell subtypes in CAR⁺ T cells after 6 days of pharmacological modulation (left) and Tn/scm enrichment (right). The bars in FIG. 3C, right graph, represent data from, EGFRt, Leu16-28z (vehicle), Leu16-28z (MYCi975), Leu16-28z (Betulin), and Leu16-28z (Rapamycin), respectively, from left to right. (D) Activation and exhaustion marker expression by CAR⁺ T cells in the absence of antigen stimulation after 6 days of pharmacological modulation. (E) Proliferation of CAR⁺ T cells stained with CellTrace Violet (CTV) dye was assayed after a 4-day culture in the absence of target cells or exogenous cytokines. The bars in FIG. 3C, right graph, FIGS. 3D, and 3E, represent data from, EGFRt, Leu16-28z (vehicle), Leu16-28z (MYCi975), Leu16-28z (Betulin), and Leu16-28z (Rapamycin), respectively, from left to right. (F) NSG mice were injected intravenously with 0.5×10⁶ ffLuc-expressing Raji cells followed by two doses of 1.35×10⁶ CAR-T cells at 7 and 12 days post tumor injection. (F) Tumor signal (in photons/sec/cm2/sr) in individual animals as quantified by bioluminescence imaging. (G) Kaplan-Meier survival curve. Log-rank (Mantel-Cox) test was performed with Holm-Sidak correction for multiple comparisons. * p<0.05, ** p<0.01, *** p<0.001, n.s. not statistically significant. Data bars indicate the means of technical triplicates±1 S.D. * p<0.05, ** p<0.01, *** p<0.001, ns not statistically significant.

FIG. 4A-C. Characterization of pharmacologically treated Leu16 CAR-T cells in vitro. (A) TNF-α production CAR+ T cells in the absence of antigen stimulation as quantified by intracellular flow cytometry. Data bars indicate the means of technical triplicates ±1 S.D. ns not statistically significant. (B) Leu16 CAR-T cell fold expansion after 6 days of inhibitor culture was tracked. The bars in FIGS. 4A and 4B, represent data from, EGFRt, Leu16-28z (vehicle), Leu16-28z (MYCi975), Leu16-28z (Betulin), and Leu16-28z (Rapamycin), respectively, from left to right. (C) A 24-hr lysis assay of CAR-T cells against Raji target cells at three effector-to-target (E:T) ratios. Data bars indicate the means of technical triplicates±1 S.D. Percent lysis was normalized to cell counts in target-only wells. In the line graph, the line that shows the least amount of % lysis represents data for EGFRt, and the lines that represent data for Leu 16 (vehicle), Leu 16 (MYCi975), Leu 16 (Betulin), and Leu16 (Rapamycin) are clustered together.

### DETAILED DESCRIPTION

### I. Administration of Therapeutic Compositions

The therapeutic cells of the disclosure may be administered by any route of administration. The cells may be administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. Any of these may be excluded in an embodiment. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. A unit dose may comprise a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

It may be desirable to have multiple administrations of the composition, e.g., 2, 3, 4, 5, 6 or more administrations (or any range derivable therein). The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The cells can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### II. Cellular Therapies

### A. Cell Culture

Cells may be cultured for at least between about 10 days and about 40 days, for at least between about 15 days and about 35 days, for at least between about 15 days and 21 days, such as for at least about 15, 16, 17, 18, 19 or 21 days. In some aspects, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days (or any range derivable therein). The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some aspects, a culture medium formulation may be explants medium (CEM) which is composed of IMDM supplemented with 10% fetal bovine serum (FBS), 100 U/ml penicillin G, 100 μg/ml streptomycin and 2 mmol/L L-glutamine. Other aspects may employ further basal media formulations, such as chosen from the ones above.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (aMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. Cells may be cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

### B. Cell Generation

Certain methods of the disclosure concern culturing the cells obtained from human tissue samples. In particular aspects of the present disclosure, cells are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain aspects cells are introduced into a culture system that features at least one substrate surface that is generally compatible with adherence of cells thereto, such that the plated cells can contact the said substrate surface, such aspects encompass plating onto a substrate, which allows adherence of cells thereto.

Cells of the present disclosure may be identified and characterized by their expression of specific marker proteins, such as cell-surface markers. Detection and isolation of these cells can be achieved, for example, through flow cytometry, ELISA, and/or magnetic beads. Reverse-transcription polymerase chain reaction (RT-PCR) may be used to quantify cell-specific genes and/or to monitor changes in gene expression in response to differentiation. In certain aspects, the marker proteins used to identify and characterize the cells are selected from the list consisting of c-Kit, Nanog, Sox2, Hey1, SMA, Vimentin, Cyclin D2, Snail, E-cadherin, Nkx2.5, GATA4, CD105, CD90, CD29, CD73, Wt1, CD34, CD45, and a combination thereof.

### C. Pharmaceutical Compositions

In certain aspects, the compositions or agents for use in the methods, such as the cell compositions, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### D. Adoptive Cell Therapy

Adoptive Cell Therapy is a form of passive immunization by the transfusion (adoptive cell transfer) of immune cells, in particular T-cells. T cells are found in blood and tissue and usually activate when they find foreign pathogens or other antigens that T-cell's surface receptors encounter parts of foreign proteins (antigens) that are displayed on surface of other cells. These latter cells can be either infected cells, or antigen presenting cells (APCs) that are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the expansion and the reinfusion of the resulting cells. Activation can take place through gene therapy, or by exposing the T cells to tumor antigens. Additional details on the preparation, selection, use, combination with other therapies, an/or administration of cells for ACT treatment are described in the literature (Cook K et al., 2018, Elahi R et al., 2018; Sharma P. et al., 2017).

In some aspects, the adoptive cell therapy comprises dendritic cell therapy, which provokes anti-tumor responses by causing dendritic cells to present tumor antigens to lymphocytes, and then activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T. One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF. Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies may include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

In some aspects, the adoptive cell therapy comprises CAR-T cell therapy. Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy. Exemplary CAR-T therapies include Tisagenlecleucel (Kymriah) and Axicabtagene ciloleucel. In some aspects, the CAR-T therapy targets CD19 or CD20.

### E. Chimeric Antigen Receptors

### 1. Signal Peptide

Polypeptides of the present disclosure may comprise a signal peptide. A “signal peptide” refers to a peptide sequence that directs the transport and localization of the protein within a cell, e.g., to a certain cell organelle (such as the endoplasmic reticulum) and/or the cell surface. In some aspects, a signal peptide directs the nascent protein into the endoplasmic reticulum. This is essential if a receptor is to be glycosylated and anchored in the cell membrane. Generally, the signal peptide natively attached to the amino-terminal most component is used (e.g., in an scFv with orientation light chain-linker-heavy chain, the native signal of the light-chain is used).

In some aspects, the signal peptide is cleaved after passage of the endoplasmic reticulum (ER), i.e., is a cleavable signal peptide. In some aspects, a restriction site is at the carboxy end of the signal peptide to facilitate cleavage.

### 2. Antigen Binding Domain

Polypeptides of the present disclosure may comprise one or more antigen binding domains. An “antigen binding domain” describes a region of a polypeptide capable of binding to an antigen under appropriate conditions. In some aspects, an antigen binding domain is a single-chain variable fragment (scFv) based on one or more antibodies (e.g., CD20 antibodies).

In some aspects, an antigen binding domain comprise a variable heavy (VH) region and a variable light (VL) region, with the VH and VL regions being on the same polypeptide. In some aspects, the antigen binding domain comprises a linker between the VH and VL regions. A linker may enable the antigen binding domain to form a desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M (or any range derivable therein). In some aspects, the KD of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M (or any derivable range therein).

Binding affinity, KA, or KD can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some aspects, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity and/or expression level in host cells (or any range derivable therein), compared to a polypeptide comprising a non-humanized binding region, such as a binding region from a mouse. In some aspects, the framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

In some aspects, the framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

Non-limiting examples of tumor antigens that may be targeted by the CAR(s) of the present disclosure include at least the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, p180erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, MUC18, LMP1, EBMA-1, BARF-1, CS1, CD319, HER1, B7H6, L1 CAM, IL6, and MET.

Tumor antigens also include, for example, a glioma-associated antigen, carcinoembryonic antigen (CEA), EGFRvlll, IL-IIRa, IL-13Ra, EGFR, FAP, B7H3, Kit, CA LX, CS-1, MUC1, BCMA, bcr-abl, HER2, b-human chorionic gonadotropin, alphafetoprotein (AFP), ALK, CD19, cyclin Bl, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewis Y, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gp100, prostein, OR51 E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1.MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, mesothelin, and any combination thereof.

Further examples of tumor cell antigens to which a CAR may be directed include at least 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CD133, CEA, c-Met, CSPG4, EGFR, EGFR family including ErbB2 (HER2), EGFRVIII, EGP2, EGP40, ERBB3, ERBB4, ErbB3/4, EPCAM, EphA2, EpCAM, folate receptor-a, FAP, FBP, fetal AchR, FR□, GD2, G250/CAIX, GD3, Glypican-3 (GPC3), GUCY2C, HER1, HER2, ICAM-1, IL-13R□2, IL-11Ra, Kras, Kras G12D, L1CAM, Lambda, Lewis-Y, Kappa, KDR, MAGE, MCSP, MET, Mesothelin, Muc1, Muc16, MUC18, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, TGF-β, WT-1, or VEGF receptors (e.g., VEGFR2), for example. The CARs may be a first, second, third, or more generation CARs. The CARs may be bispecific for any two nonidentical antigens, or they may be specific for more than two nonidentical antigens.

### 3. Peptide Spacer

A peptide spacer, such as an extracellular spacer may link an antigen-binding domain to a transmembrane domain. In some aspects, a peptide spacer is flexible enough to allow the antigen-binding domain to orient in different directions to facilitate antigen binding. In one embodiment, the spacer comprises the hinge region from IgG. In some aspects, the spacer comprises or further comprises the CH2CH3 region of immunoglobulin and portions of CD3. In some aspects, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. In some aspects, the spacer is from IgG4. An extracellular spacer may comprise a hinge region.

As used herein, the term “hinge” refers to a flexible polypeptide connector region (also referred to herein as “hinge region”) providing structural flexibility and spacing to flanking polypeptide regions and can consist of natural or synthetic polypeptides. A “hinge” derived from an immunoglobulin (e.g., IgGl) is generally defined as stretching from Glu216 to Pro230 of human IgGl (Burton (1985) Molec. Immunol., 22:161-206). Hinge regions of other IgG isotypes may be aligned with the IgGl sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425, incorporated by reference herein. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some aspects, the extracellular spacer consists of or comprises a hinge region from an immunoglobulin (e.g., IgG). Immunoglobulin hinge region amino acid sequences are known in the art; see, e.g., Tan et al. (1990) Proc. Natl. Acad. Sci. USA 87:162; and Huck et al. (1986) Nucl. Acids Res.

The length of an extracellular spacer may have effects on the CAR's signaling activity and/or the CAR-T cells' expansion properties in response to antigen-stimulated CAR signaling. In some aspects, a shorter spacer such as less than 50, 45, 40, 30, 35, 30, 25, 20, 15, 14, 13, 12, 11, or 10 amino acids is used. In some aspects, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids (or any range derivable therein) may have the advantage of increased expansion in vivo or in vitro.

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. In some aspects, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

### 4. Transmembrane Domain

Polypeptides of the present disclosure may comprise a transmembrane domain. In some aspects, a transmembrane domain is a hydrophobic alpha helix that spans the membrane. Different transmembrane domains may result in different receptor stability.

In some aspects, the transmembrane domain is interposed between the extracellular spacer and the cytoplasmic region. In some aspects, the transmembrane domain is interposed between the extracellular spacer and one or more costimulatory regions. In some aspects, a linker is between the transmembrane domain and the one or more costimulatory regions.

Any transmembrane domain that provides for insertion of a polypeptide into the cell membrane of a eukaryotic (e.g., mammalian) cell may be suitable for use. In some aspects, the transmembrane domain is derived from CD28, CD8, CD4, CD3-zeta, CD134, or CD7.

### 5. Cytoplasmic Region

After antigen recognition, receptors of the present disclosure may cluster and a signal transmitted to the cell through the cytoplasmic region. In some aspects, the costimulatory domains described herein are part of the cytoplasmic region. In some aspects, the cytoplasmic region comprises an intracellular signaling domain. An intracellular signaling domain may comprise a primary signaling domain and one or more costimulatory domains.

Cytoplasmic regions and/or costimulatory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some aspects, the cytoplasmic region includes at least one (e.g., one, two, three, four, five, six, etc.) ITAM motif as described herein. In some aspects, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2(L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2(L/I))(X3)n(YX1X2(L/I)), where n is an integer from 6 to 8, and each of the 6-8 X3 can be any amino acid.

A suitable cytoplasmic region may be an ITAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Exemplary cytoplasmic regions are known in the art. The cytoplasmic regions shown below also provide examples of regions that may be incorporated in a CAR of the disclosure:

In some aspects, a suitable cytoplasmic region can comprise an ITAM motif-containing portion of the full length DAP12 amino acid sequence. In some aspects, the cytoplasmic region is derived from FCER1G (also known as FCRG; Fc epsilon receptor I gamma chain; Fc receptor gamma-chain; fc-epsilon Rl-gamma; fcRgamma; fceRI gamma; high affinity immunoglobulin epsilon receptor subunit gamma; immunoglobulin E receptor, high affinity, gamma chain; etc.). In some aspects, a suitable cytoplasmic region can comprise an ITAM motif-containing portion of the full length FCER1G amino acid sequence.

In some aspects, the cytoplasmic region is derived from T cell surface glycoprotein CD3 delta chain (also known as CD3D; CD3-DELTA; T3D; CD3 antigen, delta subunit; CD3 delta; CD3□; CD3d antigen, delta polypeptide (TiT3 complex); OKT3, delta chain; T cell receptor T3 delta chain; T cell surface glycoprotein CD3 delta chain; etc.). In some aspects, a suitable cytoplasmic region can comprise an ITAM motif-containing portion of the full length CD3 delta amino acid sequence. In some aspects, the cytoplasmic region is derived from T cell surface glycoprotein CD3 epsilon chain (also known as CD3e, CD3□; T cell surface antigen T3/Leu-4 epsilon chain, T cell surface glycoprotein CD3 epsilon chain, AI504783, CD3, CD3-epsilon, T3e, etc.). In some aspects, a suitable cytoplasmic region can comprise an ITAM motif-containing portion of the full length CD3 epsilon amino acid sequence. In some aspects, the cytoplasmic region is derived from T cell surface glycoprotein CD3 gamma chain (also known as CD3G, CD3y, T cell receptor T3 gamma chain, CD3-GAMMA, T3G, gamma polypeptide (TiT3 complex), etc.). In some aspects, a suitable cytoplasmic region can comprise an ITAM motif-containing portion of the full length CD3 gamma amino acid sequence. In some aspects, the cytoplasmic region is derived from T cell surface glycoprotein CD3 zeta chain (also known as CD3Z, CD3ζ, T cell receptor T3 zeta chain, CD247, CD3-ZETA, CD3H, CD3Q, T3Z, TCRZ, etc.). In some aspects, a suitable cytoplasmic region can comprise an ITAM motif-containing portion of the full length CD3 zeta amino acid sequence.

In some aspects, the cytoplasmic region is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In some aspects, a suitable cytoplasmic region can comprise an ITAM motif-containing portion of the full length CD79A amino acid sequence.

### 6. Costimulatory Region

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM. It is specifically contemplated that any of these may be excluded from an embodiment.

A costimulatory region may have a length of at least, at most, or exactly 20, 25, 30, 35, 40, 50, 60, 70, 80, 90, 100, 150, 200, or 300 amino acids or any range derivable therein. In some aspects, the costimulatory region is derived from an intracellular portion of the transmembrane protein 4-1BB (also known as TNFRSF9; CD137; CDw137; ILA; etc.). In some aspects, the costimulatory region is derived from an intracellular portion of the transmembrane protein CD28 (also known as Tp44). In some aspects, the costimulatory region is derived from an intracellular portion of the transmembrane protein ICOS (also known as AILIM, CD278, and CVID1). In some aspects, the costimulatory region is derived from an intracellular portion of the transmembrane protein OX-40 (also known as TNFRSF4, RP5-902P8.3, ACT35, CD134, OX40, TXGP1L). In some aspects, the costimulatory region is derived from an intracellular portion of the transmembrane protein BTLA (also known as BTLA1 and CD272). In some aspects, the costimulatory region is derived from an intracellular portion of the transmembrane protein CD27 (also known as S 152, T14, TNFRSF7, and Tp55). In some aspects, the costimulatory region is derived from an intracellular portion of the transmembrane protein CD30 (also known as TNFRSF8, D1S166E, and Ki-1). In some aspects, the costimulatory region is derived from an intracellular portion of the transmembrane protein GITR (also known as TNFRSF18, RP5-902P8.2, AITR, CD357, and GITR-D). In some aspects, the costimulatory region derived from an intracellular portion of the transmembrane protein HVEM (also known as TNFRSF14, RP3-395M20.6, ATAR, CD270, HVEA, HVEM, LIGHTR, and TR2).

### 7. Detection Peptides

In some aspects, the polypeptides described herein may further comprise a detection peptide. Suitable detection peptides include hemagglutinin (HA; e.g., YPYDVPDYA (SEQ ID NO: 41); FLAG (e.g., DYKDDDDK (SEQ ID NO:42); c-myc (e.g., EQKLISEEDL; SEQ ID NO: 43), and the like. Other suitable detection peptides are known in the art.

### 8. Peptide Linkers

In some aspects, the polypeptides of the disclosure include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Example flexible linkers include glycine polymers (G) n, glycine-serine polymers (including, for example, (GS)n, (GSGGS)n (SEQ ID NO:44), (G4S)n and (GGGS)n (SEQ ID NO: 45), where n is an integer of at least one. In some aspects, n is at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO:46), GGSGG (SEQ ID NO:47), GSGSG (SEQ ID NO:48), GSGGG (SEQ ID NO:49), GGGSG (SEQ ID NO:50), or GSSSG (SEQ ID NO:51).

### F. Cells

Certain aspects relate to cells comprising polypeptides or nucleic acids of the disclosure. In some aspects the cell is an immune cell or a T cell. “T cell” includes all types of immune cells expressing CD3 including T-helper cells, invariant natural killer T (iNKT) cells, cytotoxic T cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### III. Treatment of Disease

Methods may be employed with respect to individuals who have tested positive for such disorders or who are deemed to be at risk for developing such a condition or related condition. In some aspects, the compositions and methods described herein are used to treat an inflammatory or autoimmune component of a disorder listed herein and/or known in the art.

Certain aspects of the disclosure relate to the treatment of cancer and/or use of cancer antigens. The cancer to be treated or antigen may be an antigen associated with any cancer known in the art or, for example, epithelial cancer, (e.g., breast, gastrointestinal, lung), prostate cancer, bladder cancer, lung (e.g., small cell lung) cancer, colon cancer, ovarian cancer, brain cancer, gastric cancer, renal cell carcinoma, pancreatic cancer, liver cancer, esophageal cancer, head and neck cancer, or a colorectal cancer. In some aspects, the cancer to be treated or antigen is from one of the following cancers: adenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, meningiosarcoma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, ovarian cancer, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), or Meigs' syndrome. Any of these cancers or conditions may be excluded in an embodiment.

### IV. Biological Samples

Methods may include obtaining a sample from a subject and/or the subject may be one that has had a biological sample tested in a method step described herein. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from esophageal tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### V. Gene Transfer Techniques

### A. Gene Editing

In certain aspects, engineered nucleases may be used to introduce transgenes into cells, such as progenitor cells, stem cells, HSPCs, ES cells, iPSCs, and human embryonic mesodermal progenitor cells. In aspects wherein cells are genetically modified, such as to add or reduce one or more features, the genetic modification may occur by any suitable method. For example, any genetic modification compositions or methods may be used to introduce exogenous nucleic acids into cells or to edit the genomic DNA, such as gene editing, homologous recombination or non-homologous recombination, RNA-mediated genetic delivery or any conventional nucleic acid delivery methods. In some aspects, the gene transfer technique comprises targeted integration into an endogenous locus of the cell's genome, such as into the granzyme A locus. Non-limiting examples of the genetic modification methods may include gene editing methods such as by CRISPR/CAS9, zinc finger nuclease, or TALEN technology.

Genome editing, or genome editing with engineered nucleases (GEEN) is a type of genetic engineering in which DNA is inserted, replaced, or removed from a genome using artificially engineered nucleases, or “molecular scissors.” The nucleases create specific double-stranded break (DSBs) at desired locations in the genome, and harness the cell's endogenous mechanisms to repair the induced break by natural processes of homologous recombination (HR) and nonhomologous end-joining (NHEJ).

Non-limiting engineered nucleases include: Zinc finger nucleases (ZFNs), Transcription Activator-Like Effector Nucleases (TALENs), the CRISPR/Cas9 system, and engineered meganuclease re-engineered homing endonucleases. Any of the engineered nucleases known in the art can be used in certain aspects of the methods and compositions.

It is commonly practiced in genetic analysis that in order to understand the function of a gene or a protein function one interferes with it in a sequence-specific way and monitors its effects on the organism. However, in some organisms it is difficult or impossible to perform site-specific mutagenesis, and therefore more indirect methods have to be used, such as silencing the gene of interest by short RNA interference (siRNA). Yet gene disruption by siRNA can be variable and incomplete. Genome editing with nucleases such as ZEN is different from siRNA in that the engineered nuclease is able to modify DNA-binding specificity and therefore can in principle cut any targeted position in the genome, and introduce modification of the endogenous sequences for genes that are impossible to specifically target by conventional RNAi. Furthermore, the specificity of ZFNs and TALENs are enhanced as two ZFNs are required in the recognition of their portion of the target and subsequently direct to the neighboring sequences.

Meganucleases, found commonly in microbial species, have the unique property of having very long recognition sequences (>14 bp) thus making them naturally very specific. This can be exploited to make site-specific DSB in genome editing; however, the challenge is that not enough meganucleases are known, or may ever be known, to cover all possible target sequences. To overcome this challenge, mutagenesis and high throughput screening methods have been used to create meganuclease variants that recognize unique sequences. Others have been able to fuse various meganucleases and create hybrid enzymes that recognize a new sequence. Yet others have attempted to alter the DNA interacting amino acids of the meganuclease to design sequence specific meganucleases in a method named rationally designed meganuclease (U.S. Pat. No. 8,021,867 B2, incorporated herein by reference).

Meganuclease have the benefit of causing less toxicity in cells compared to methods such as ZENs likely because of more stringent DNA sequence recognition; however, the construction of sequence specific enzymes for all possible sequences is costly and time consuming as one is not benefiting from combinatorial possibilities that methods such as ZENs and TALENs utilize. So there are both advantages and disadvantages.

As opposed to meganucleases, the concept behind ZFNs and TALENs is more based on a non-specific DNA cutting enzyme which would then be linked to specific DNA sequence recognizing peptides such as zinc fingers and transcription activator-like effectors (TALEs). One way was to find an endonuclease whose DNA recognition site and cleaving site were separate from each other, a situation that is not common among restriction enzymes. Once this enzyme was found, its cleaving portion could be separated which would be very non-specific as it would have no recognition ability. This portion could then be linked to sequence recognizing peptides that could lead to very high specificity. An example of a restriction enzyme with such properties is FokI. Additionally FokI has the advantage of requiring dimerization to have nuclease activity and this means the specificity increases dramatically as each nuclease partner would recognize a unique DNA sequence. To enhance this effect, FokI nucleases have been engineered that can only function as heterodimers and have increased catalytic activity. The heterodimer functioning nucleases would avoid the possibility of unwanted homodimer activity and thus increase specificity of the DSB.

Although the nuclease portion of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Zinc fingers have been more established in these terms and approaches such as modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries among other methods have been used to make site specific nucleases.

### B. Gene Delivery

In certain aspects, vectors could be constructed to comprise transgene nucleic acid sequences for genetic modification of any cells used herein, particularly the starting cells, such as stem or progenitor cells induced to differentiate into mature cells. Details of components of these vectors and delivery methods are disclosed below.

The cells in certain aspects can be made to contain one or more genetic alterations by genetic engineering of the cells either before or after differentiation (US 2002/0168766). A cell is said to be “genetically altered”, “genetically modified” or “transgenic” when an exogenous nucleic acid or polynucleotide has been transferred into the cell by any suitable means of artificial manipulation, or where the cell is a progeny of the originally altered cell that has inherited the polynucleotide. For example, the cells can be processed to increase their replication potential by genetically altering the cells to express telomerase reverse transcriptase, either before or after they progress to restricted developmental lineage cells or terminally differentiated cells (US 2003/0022367).

### C. Vector

One of skill in the art would be well equipped to construct a vector through standard recombinant techniques (see, for example, Maniatis et al., 1988 and Ausubel et al., 1994, both incorporated herein by reference).

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

### 1. Regulatory Elements

Eukaryotic expression cassettes included in the vectors particularly contain (in a 5′-to-3′ direction) a eukaryotic transcriptional promoter operably linked to a protein-coding sequence, splice signals including intervening sequences, and a transcriptional termination/polyadenylation sequence.

### 2. Promoter/Enhancers

A “promoter” is a control sequence that is a region of a nucleic acid sequence at which initiation and rate of transcription are controlled. In aspects of the disclosure, the promoter or enhancer may include a promoter or enhancer from the Granzyme A gene. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

The promoter region of the disclosure may be an endogenous promoter region. An endogenous promoter region refers to the situation in which the promoter region is in it's endogenous genomic setting, such that the sequences upstream of the promoter (i.e. 5′ region) are the substantially the same as those that are in the wild-type cell. Substantially the same could refer to a region that is at least 80, 85, 90, 95, 96, 97, 98, or 99% identical to the upstream region of the wild-type. An endogenous promoter region also refers to a situation in which the promoter is in the same genomic location as the wild-type promoter. In some aspects, the endogenous promoter refers to a promoter in a cell this is unmodified or that is or is at least 70, 75, 80, 85, 90, 95, 96, 97, 98, or 99% identical to the wild-type promoter.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 110 bp upstream of the start site, although a number of promoters have been shown to contain functional elements downstream of the start site as well. To bring a coding sequence “under the control of” a promoter, one positions the 5 end of the transcription initiation site of the transcriptional reading frame “downstream” of (i.e., 3 of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

### 3. Protease Cleavage Sites/Self-Cleaving Peptides and Internal Ribosome Binding Sites

Suitable protease cleavages sites and self-cleaving peptides are known to the skilled person (see, e.g., in Ryan et al., 1997; Scymczak et al., 2004). Examples of protease cleavage sites are the cleavage sites of potyvirus NIa proteases (e.g. tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PY\IF (parsnip yellow fleck virus) 3C-like protease, thrombin, factor Xa and enterokinase. Due to its high cleavage stringency, TEV (tobacco etch virus) protease cleavage sites may be used.

Exemplary self-cleaving peptides (also called “cis-acting hydrolytic elements”, CHYSEL; see deFelipe (2002) are derived from potyvirus and cardiovirus 2A peptides. Particular self-cleaving peptides may be selected from 2A peptides derived from FMDV (foot-and-mouth disease virus), equine rhinitis A virus, Thoseà asigna virus and porcine teschovirus.

A specific initiation signal also may be used for efficient translation of coding sequences in a polycistronic message. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain aspects, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

### 4. Termination Signals

The vectors or constructs may comprise at least one termination signal. A “termination signal” or “terminator” is comprised of the DNA sequences involved in specific termination of an RNA transcript by an RNA polymerase. Thus, in certain aspects a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other aspects involving eukaryotes, the terminator comprises a signal for the cleavage of the RNA, and the terminator signal promotes polyadenylation of the message. The terminator and/or polyadenylation site elements can serve to enhance message levels and to minimize read through from the cassette into other sequences.

Terminators contemplated include any known terminator of transcription described herein or known to one of ordinary skill in the art, including but not limited to, for example, the termination sequences of genes, such as for example the bovine growth hormone terminator or viral termination sequences, such as for example the SV40 terminator. In certain aspects, the termination signal may be a lack of transcribable or translatable sequence, such as due to a sequence truncation.

### 5. Polyadenylation Signals

In expression, particularly eukaryotic expression, one will typically include a polyadenylation signal to effect proper polyadenylation of the transcript. The nature of the polyadenylation signal is not believed to be crucial to the successful practice, and any such sequence may be employed. Exemplary aspects include the SV40 polyadenylation signal or the bovine growth hormone polyadenylation signal, convenient and known to function well in various target cells. Polyadenylation may increase the stability of the transcript or may facilitate cytoplasmic transport.

### D. Vector Delivery

Genetic modification or introduction of transgene nucleic acids into cells of the disclosure may use any suitable methods for nucleic acid delivery for transformation of a cell, as described herein or as would be known to one of ordinary skill in the art. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985), and any combination of such methods. Through the application of techniques such as these, organelle(s), cell(s), tissue(s) or organism(s) may be stably or transiently transformed.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g., Sambrook et al. (2001) Molecular Cloning: A Laboratory Manual, Cold Spring Harbor Laboratory, New York). One illustrative, but non-limiting method for the introduction of a polynucleotide into a host cell is calcium phosphate transfection.

Biological methods for introducing a polynucleotide of interest into a host cell can include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g. U.S. Pat. Nos. 5,350,674 and 5,585,362, and the like).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An illustrative colloidal system for use as a delivery vehicle in vitro and in vivo is a liposome (e.g., an artificial membrane vesicle).

### 1. Liposome Mediated Transfection

In the case where a non-viral delivery system is utilized, one illustrative delivery vehicle is a lipid and/or a liposome. The use of lipid formulations is contemplated for the introduction of the nucleic acids into a host cell (in vitro, ex vivo, or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In a certain aspect, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self rearrangement before the formation of closed structures and entrap water and dissolved solutes between the lipid bilayers (Ghosh and Bachhawat, 1991). Also contemplated is a nucleic acid complexed with Lipofectamine (Gibco BRL) or Superfect (Qiagen). The amount of liposomes used may vary upon the nature of the liposome as well as the cell used, for example, about 5 to about 20 □g vector DNA per 1 to 10 million of cells may be contemplated.

Liposome mediated nucleic acid delivery and expression of foreign DNA in vitro has been very successful (Nicolau and Sene, 1982; Fraley et al., 1979; Nicolau et al., 1987). The feasibility of liposome mediated delivery and expression of foreign DNA in cultured chick embryo, HeLa and hepatoma cells has also been demonstrated (Wong et al., 1980).

In certain aspects, a liposome may be complexed with a hemagglutinating virus (HVJ). This has been shown to facilitate fusion with the cell membrane and promote cell entry of liposome encapsulated DNA (Kaneda et al., 1989). In other aspects, a liposome may be complexed or employed in conjunction with nuclear non histone chromosomal proteins (HMG 1) (Kato et al., 1991). In yet further aspects, a liposome may be complexed or employed in conjunction with both HVJ and HMG 1. In other aspects, a delivery vehicle may comprise a ligand and a liposome.

In various aspects lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform can be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al. (1991) Glycobiology 5:505-510). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### 2. Electroporation

In certain aspects, a nucleic acid is introduced into a cell via electroporation. Electroporation involves the exposure of a suspension of cells and DNA to a high voltage electric discharge. Recipient cells can be made more susceptible to transformation by mechanical wounding. Also the amount of vectors used may vary upon the nature of the cells used, for example, about 5 to about 20 □g vector DNA per 1 to 10 million of cells may be contemplated.

Transfection of eukaryotic cells using electroporation has been quite successful. Mouse pre B lymphocytes have been transfected with human kappa immunoglobulin genes (Potter et al., 1984), and rat hepatocytes have been transfected with the chloramphenicol acetyltransferase gene (Tur Kaspa et al., 1986) in this manner.

### 3. Calcium Phosphate

In other aspects, a nucleic acid is introduced to the cells using calcium phosphate precipitation. Human KB cells have been transfected with adenovirus 5 DNA (Graham and Van Der Eb, 1973) using this technique. Also in this manner, mouse L (A9), mouse C127, CHO, CV 1, BHK, NIH3T3 and HeLa cells were transfected with a neomycin marker gene (Chen and Okayama, 1987), and rat hepatocytes were transfected with a variety of marker genes (Rippe et al., 1990).

### 4. DEAE Dextran

In another aspect, a nucleic acid is delivered into a cell using DEAE dextran followed by polyethylene glycol. In this manner, reporter plasmids were introduced into mouse myeloma and erythroleukemia cells (Gopal, 1985).

### VI. Nucleic Acids and Polypeptides

### A. Nucleic Acids

In certain aspects, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### 1. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### 2. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### 3. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or part of one or more of the CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### B. Polypeptides

As used herein, a “protein” “peptide” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 1000, 1200, 1400, 1600, 1800, or 2000 amino acid residues or nucleic acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.).

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS: 1-51. In specific aspects, the peptide or polypeptide is or is based on a human sequence. In certain aspects, the peptide or polypeptide is not naturally occurring and/or is in a combination of peptides or polypeptides.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 of any of SEQ ID NOS: 1-51 (or any derivable range therein) and may be a substitution with any amino acid or may be a substitution with a alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, the protein, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS: 1-51.

In some aspects, the protein, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS: 1-51 and have or have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOS: 1-51.

In some aspects, the protein, polypeptide, or nucleic acid may comprise, comprise at least, or comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids or nucleic acids of SEQ ID NOS: 1-51.

In some aspects, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids of SEQ ID NOS: 1-51 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOS: 1-51.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 of any of SEQ ID NOS: 1-51 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 (or any derivable range therein) contiguous amino acids or nucleotides of any of SEQ ID NOS: 1-51.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein structure.

In some aspects of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the antibody that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

### VII. Sequences

| | | SEQ |
|---|---|---|
| | | ID |
| Description | Sequence | NO: |
| Leu16 VL | DIVLTQSPAILSASPGEKVTMTCRASSSVNYMDWYQKKPG | 1 |
| | SSPKPWIYATSNLASGVPARFSGSGSGTSYSLTISRVEAEDA | |
| | ATYYCQQWSFNPPTFGGGTKLEIK | |
| Leu16 VH | EVQLQQSGAELVKPGASVKMSCKASGYTFTSYNMHWVKQ | 2 |
| | TPGQGLEWIGAIYPGNGDTSYNQKFKGKATLTADKSSSTA | |
| | YMQLSSLTSEDSADYYCARSNYYGSSYWFFDVWGAGTTV | |
| | TVSS | |
| Rituximab VL | QIVLSQSPAILSASPGEKVTMTCRASSSVSYIHWFQQKPGSS | 3 |
| | PKPWIYATSNLASGVPVRFSGSGSGTSYSLTISRVEAEDAAT | |
| | YYCQQWTSNPPTFGGGTKLEIK | |
| Rituximab VH | QVQLQQPGAELVKPGASVKMSCKASGYTFTSYNMHWVK | 4 |
| | QTPGRGLEWIGAIYPGNGDTSYNQKFKGKATLTADKSSST | |
| | AYMQLSSLTSEDSAVYYCARSTYYGGDWYFNVWGAGTT | |
| | VTVSS | |
| Leu16 HCDR1 | SYNMH | 5 |
| Leu16 HCDR2 | AIYPGNGDTSYNQKFKG | 6 |
| Leu16 HCDR3 | SNYYGSSYWFFDV | 7 |
| Leu16 LCDR1 | RASSSVNYMD | 8 |
| Leu16 LCDR2 | ATSNLAS | 9 |
| Leu16 LCDR3 | QQWSFNPPT | 10 |
| Leu16 HFR1 | EVQLQQSGAELVKPGASVKMSCKASGYTFT | 11 |
| Leu16 HFR2 | WVKQTPGQGLEWIG | 12 |
| Leu16 HFR3 | KATLTADKSSSTAYMQLSSLTSEDSADYYCAR | 13 |
| Leu16 HFR4 | WGAGTTVTVSS | 14 |
| Leu16 LFR1 | DIVLTQSPAILSASPGEKVTMTC | 15 |
| Leu16 LFR2 | WYQKKPGSSPKPWIY | 16 |
| Leu16 LFR3 | GVPARFSGSGSGTSYSLTISRVEAEDAATYYC | 17 |
| Leu16 LFR4 | FGGGTKLEIK | 18 |
| Rituximab | SYNMH | 19 |
| HCDR1 | | |
| Rituximab | AIYPGNGDTSYNQKFKG | 20 |
| HCDR2 | | |
| Rituximab | STYYGGDWYFNV | 21 |
| HCDR3 | | |
| Rituximab | RASSSVSYIH | 22 |
| LCDR1 | | |
| Rituximab | ATSNLAS | 23 |
| LCDR2 | | |
| Rituximab | QQWTSNPPT | 24 |
| LCDR3 | | |
| Rituximab | QVQLQQPGAELVKPGASVKMSCKASGYTFT | 25 |
| HFR1 | | |
| Rituximab | WVKQTPGRGLEWIG | 26 |
| HFR2 | | |
| Rituximab | KATLTADKSSSTAYMQLSSLTSEDSAVYYCAR | 27 |
| HFR3 | | |
| Rituximab | WGAGTTVTVSS | 28 |
| HFR4 | | |
| Rituximab | QIVLSQSPAILSASPGEKVTMTC | 29 |
| LFR1 | | |
| Rituximab | WFQQKPGSSPKPWIY | 30 |
| LFR2 | | |
| Rituximab | GVPVRFSGSGSGTSYSLTISRVEAEDAATYYC | 31 |
| LFR3 | | |
| Rituximab | FGGGTKLEIK | 32 |
| LFR4 | | |
| Murine kappa | METDTLLLWVLLLWVPGSTG | 33 |
| chain signal | | |
| sequence | | |
| scFv Linker | GSTSGGGSGGGSGGGGSS | 34 |
| IgG4 | ESKYGPPCPPCPAPEFEGGPSVFLFPPKPKDTLMISRTPEVTC | 35 |
| hinge/CH2/CH3 | VVVDVSQEDPEVQFNWYVDGVEVHNAKTKPREEQFQSTY | |
| | RVVSVLTVLHQDWLNGKEYKCKVSNKGLPSSIEKTISKAK | |
| | GQPREPQVYTLPPSQEEMTKNQVSLTCLVKGFYPSDIAVE | |
| | WESNGQPENNYKTTPPVLDSDGSFFLYSRLTVDKSRWQEG | |
| | NVFSCSVMHEALHNHYTQKSLSLSLGK | |
| CD28 | MFWVLVVVGGVLACYSLLVTVAFIIFWV | 36 |
| transmembrane | | |
| CD28 | RSKRSRGGHSDYMNMTPRRPGPTRKHYQPYAPPRDFAAY | 37 |
| cytoplasmic | RS | |
| (gg) | | |
| CD3-zeta | RVKFSRSADAPAYQQGQNQLYNELNLGRREEYDVLDKRR | 38 |
| cytoplasmic | GRDPEMGGKPRRKNPQEGLYNELQKDKMAEAYSEIGMKG | |
| | ERRRGKGHDGLYQGLSTATKDTYDALHMQALPPR | |
| T2A | LEGGGEGRGSLLTCGDVEENPGPR | 39 |
| EGFRt | MLLLVTSLLLCELPHPAFLLIPRKVCNGIGIGEFKDSLSINAT | 40 |
| | NIKHFKNCTSISGDLHILPVAFRGDSFTHTPPLDPQELDILKT | |
| | VKEITGFLLIQAWPENRTDLHAFENLEIIRGRTKQHGQFSLA | |
| | VVSLNITSLGLRSLKEISDGDVIISGNKNLCYANTINWKKLF | |
| | GTSGQKTKIISNRGENSCKATGQVCHALCSPEGCWGPEPRD | |
| | CVSCRNVSRGRECVDKCNLLEGEPREFVENSECIQCHPECL | |
| | PQAMNITCTGRGPDNCIQCAHYIDGPHCVKTCPAGVMGEN | |
| | NTLVWKYADAGHVCHLCHPNCTYGCTGPGLEGCPTNGPK | |
| | IPSIATGMVGALLLLLVVALGIGLFM | |

### VIII. Examples

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Example 1: Pharmacological Modulation of Chimeric Antigen Receptor (CAR)-T Cell Signaling Pathways to Improve Tumor-Killing Efficacy

Chimeric antigen receptors (CARs) are fusion proteins whose functional domains are often connected in a plug-and-play manner to generate multiple CAR variants. However, CARs with highly similar sequences can nonetheless exhibit dramatic differences in function, and approaches to rationally optimize CAR proteins are critical to the development of effective CART cell therapies. Using tonic signaling as a guide in rational protein design, the inventors identified phosphoinositide 3-kinase/protein kinase B (PI3K/AKT) signaling as the primary driver of CD20 CAR tonic signaling through transcriptomic analyses. Pharmacological modulation of CAR tonic signaling demonstrated a causal role between high levels of CAR tonic signaling and poor in vivo anti-tumor efficacy. In fact, anti-tumor efficacy can be enhanced through pharmacological minimization of CAR tonic signaling, irrespective of whether the CAR has a high or low proclivity for tonic signaling.

In this example, the inventors demonstrate that tonic signaling can occur even when CARs do not self-aggregate, and that the intensity of tonic signaling can be modulated by pharmacological inhibition of PI3K/AKT signaling. Minimization of CAR tonic signaling through AKT or mechanistic target of rapamycin (mTOR) inhibition provided a straightforward means to enhance CAR-T cell efficacy. However, contrary to expectations, the inventors' results also indicate that CAR-T cell optimization is not only achieved by simple minimization of tonic signaling, but rather by programming T cells to effectively transition from their resting state to a productively stimulated state, characterized by enrichment of highly functional memory T cells upon antigen exposure. Collectively, these findings demonstrate pharmacological modulation of CAR-T cells as a means to modulate CAR-T cell tonic signaling.

### A. Methods

Construction of anti-CD20 scFvs and CARs. Anti-CD20 CARs were constructed by assembling an scFv (in VL-VH orientation), an extracellular IgG4 hinge-CH2-CH3 spacer containing the L235E N297Q mutation, CD28 transmembrane and cytoplasmic domain, CD3z cytoplasmic domain, and a T2A “self-cleaving” sequence followed by a truncated epidermal growth factor receptor (EGFRt) with the MSCV backbone. EGFRt was used as a transduction and sorting marker. The abovementioned anti-CD20 CAR constructs were used as templates to generate CAR-HaloTag fusion proteins for microscopy imaging of CAR clustering.

Cell line generation and maintenance. HEK 293T and Raji cells were obtained from ATCC. Leu16 and rituximab scFv-expressing HEK293T cell lines were generated by retroviral transduction of HEK293T cells to express each scFv fused with EGFP via a 2A peptide, and EGFP+ cells were sorted by fluorescence-activated cell sorting (FACS) on a FACSAria (II) cell sorter (BD Bioscience) at the UCLA Flow Cytometry Core Facility. CD20+K562 cells were generated by transduction of K562 cells with a retroviral construct encoding full-length CD20. K562 cells with varying CD20 expression levels were generated by FACS sorting of CD20+K562 into bins of different antigen densities. CHLA-255-Luc-EGFP cells were generated by retroviral transduction of CHLA-255-Luc to express EGFP, and EGFP+ cells were enriched by FACS. HEK 293T cells were cultured in DMEM (HyClone) supplemented with 10% heat-inactivated FBS (HI-FBS; ThermoFisher). CHLA-255-Luc-EGFP cells were cultured in IMDM (ThermoFisher) with 10% HI-FBS. Primary human T cells, Raji, and K562 cells were cultured in RPMI-1640 (Lonza) with 10% HI-FBS. For CAR-T cells used in metabolomics studies, T cells were cultured in RPMI-1640 containing 2 g/L of 1,2-13C-glucose with 10% heat-inactivated dialyzed FBS (HI-dFBS).

Retrovirus production and generation of human primary CAR-T cells. Retroviral supernatants were produced by transient co-transfection of HEK 293T cells with pRD114/pHIT60 virus-packaging plasmids and plasmids encoding CARs or control constructs using linear polyethylenimine (PEI, 25 kDa; Polysciences). Supernatants were collected 48 and 72 hours later and pooled after removal of cell debris by a 0.45 μm membrane filter. Healthy donor blood was obtained from the UCLA Blood and Platelet Center. CD8⁺ T cells were isolated using RosetteSep Human CD8⁺ T Cell Enrichment Cocktail (StemCell Technologies) following manufacturer's protocol. Peripheral blood mononuclear cells (PBMCs) were isolated from a Ficoll-Paque PLUS (GE Healthcare) density gradient. CD14⁻/CD25⁻/CD62L⁺ naïve/memory T cells (TN/M) were enriched from PBMCs using magnetic-activated cell sorting (MACS; Miltenyi). TN/M cells were stimulated with CD3/CD28 Dynabeads (ThermoFisher) at a 3:1 cell-to-bead ratio on Day 0 (day of isolation) and transduced with retroviral supernatant on Day 2 and Day 3. Dynabeads were removed on Day 7. T cells were cultured in T-cell media (RPMI-1640 supplemented with 10% HI-FBS) and fed with recombinant human IL-2 (ThermoFisher) and IL-15 (Miltenyi) every 2 days to final concentrations of 50 U/mL and 1 ng/ml, respectively. For CAR-T cells used in RNA-seq and metabolomics studies, T cells were enriched for CAR⁺ expression by magnetic cell sorting via staining of EGFRt with biotinylated cetuximab (Eli Lilly; biotinylated in-house) followed by anti-biotin microbeads (Miltenyi).

For inhibitor analysis, starting on Day 8, T cells were cultured in T-cell media with cytokine support in the presence of 1 μM PI3K8 isoform inhibitor (CAL-101, Apex Biotechnology #A300520)), 1 μM AKT inhibitor (AZD5363, Selleckchem #S8019), 100 nM mTORC1 inhibitor (Rapamycin, LC Laboratories #R-5000), 2 μM c-Myc inhibitor (MYCi975, MedChemExpress #HY-129601), SREBP inhibitor (Betulin, MilliporeSigma #92648), or DMSO as a vehicle control. Each inhibitor molecule was solubilized in DMSO.

Cytokine production quantification by ELISA. In 96-well U-bottom plates, 5×10⁵ CAR⁺ T cells were incubated with 2.5×10⁵ EGFP-expressing parental K562 (CD19 CD20) or CD19⁺CD20⁺ K562 target cells at a 2:1 effector-to-target (E:T) ratio. To control for cell density while accounting for differences in transduction efficiency, untransduced T cells were added as necessary to reach the same number of total T cells per well. After a 48-hour co-incubation, cells were spun down at 300× g for 2 min. Supernatant was harvested and cytokine levels were quantified by ELISA (BioLegend).

Proliferation assay. T cells were stained with 1.25 μM CellTrace Violet (ThermoFisher) and 4×10⁵ CAR⁺ T cells were seeded in each well in 96-well U-bottom plates. Untransduced T cells were added to wells as needed to normalize for differing transduction efficiencies and ensure the total number of T cells per well was consistent throughout. Cultures were passaged as needed, and CTV dilution was analyzed on a MACSQuant VYB flow cytometer after a 4-day co-incubation.

Cytotoxicity assay with repeated antigen challenge. CAR⁺ T cells were seeded at 4×10⁵ cell/well in 24-well plate and coincubated with target cells at a 2:1 E:T ratio. Untransduced T cells were added to wells as needed to normalize for differing transduction efficiencies and ensure the total number of T cells per well was consistent throughout. Cell counts were quantified by a MACSQuant VYB flow cytometer every 2 days prior to addition of fresh target cells (2×10⁵ cells/well).

Cytotoxicity assay with Raji cells. Raji cells were seeded at 2×10⁵ cells/well in 96-well U-bottom plate and coincubated with CAR⁺ T cells at 1:1, 3.33:1, and 10:1 E:T ratios. Untransduced T cells were added to wells as needed to normalize for differing transduction efficiencies and ensure the total number of T cells per well was consistent throughout. Remaining target cells were quantified by MACSQuant VYB flow cytometer 24-hr post co-incubation.

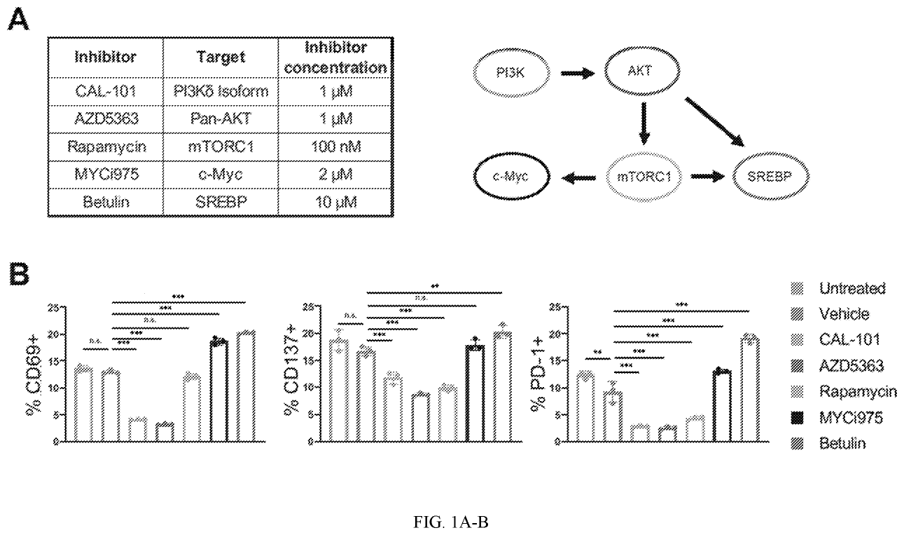

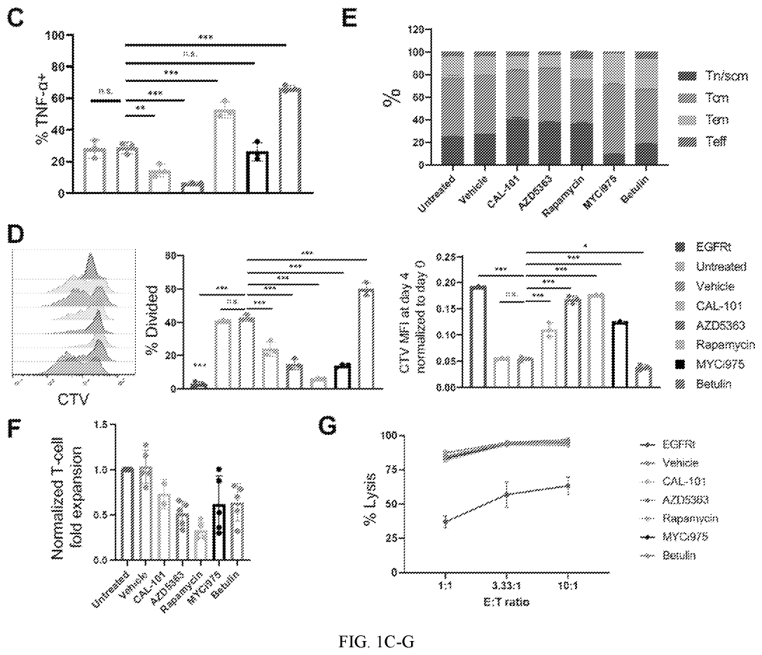

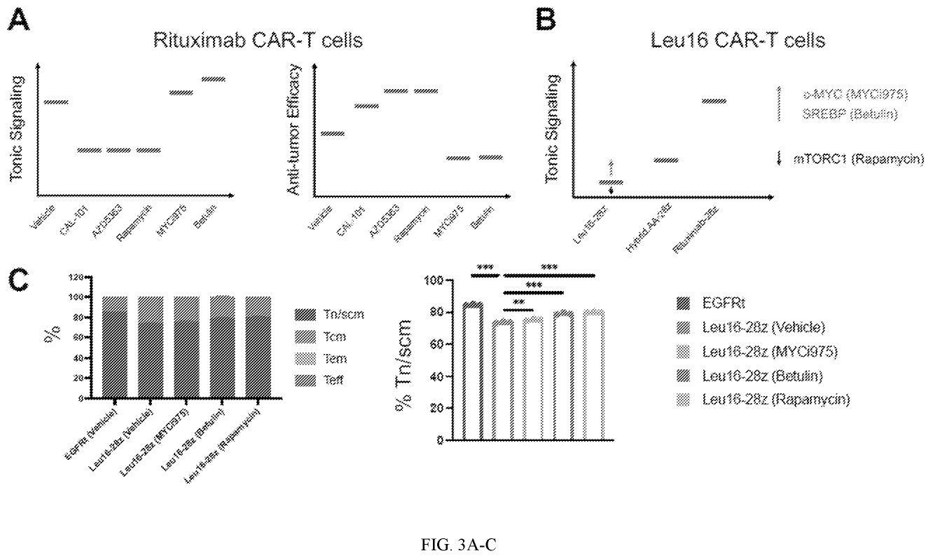

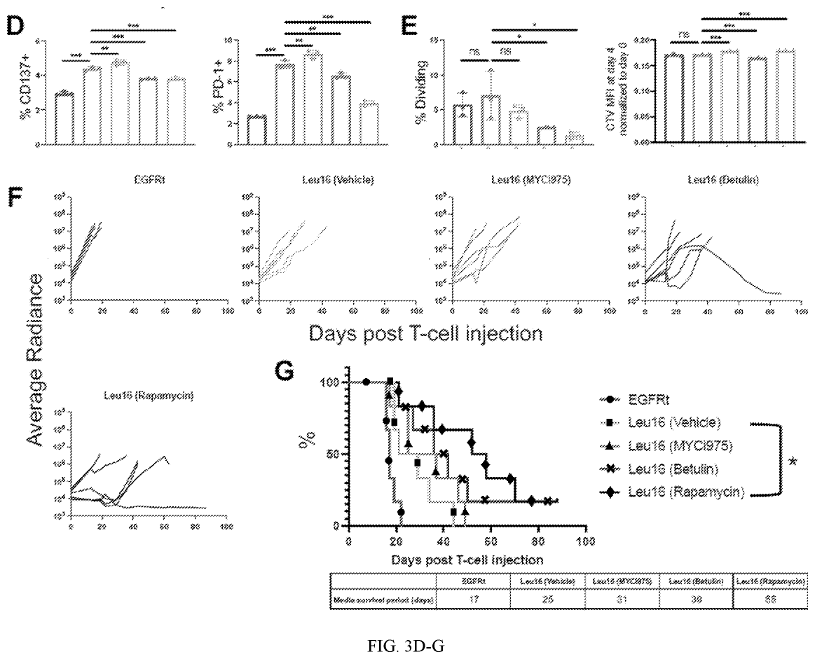

Antibody staining for flow-cytometry analysis. EGFRt expression was measured with biotinylated cetuximab (Eli Lilly; biotinylated in-house), followed by PE-conjugated streptavidin (Jackson ImmunoResearch #016-110-084). CAR expression was quantified by surface epitope staining using anti-Fc (Alexa Fluor 488, Jackson ImmunoResearch #709-546-098). Antigen-independent activation-marker expression of CAR-T cells was evaluated by antibody staining for CD137 (PE/Cy7, clone 4B4-1, BioLegend #309818), and PD-1 (FITC, clone EH12.2H7, BioLegend #329904) on Days 18 (i.e., 18 days after Dynabead addition and 11 days after Dynabead removal) and after 5-6 days of inhibitor culture (FIG. 1, FIG. 3). T-cell subtype of CAR-T cells was evaluated by antibody staining for CD45RO (VioBlue®, clone REA611, Miltenyi #130-119-620) and CD62L (APC, clone DREG56, Invitrogen #17-0629-42) after 5-6 days of inhibitor culture (FIG. 1, FIG. 3). T-cell persistence in vivo was monitored by antibody staining of retro-orbital blood, liver, and spleen samples. Samples were treated with red blood cell lysis solution (10×, Miltenyi) following manufacturer's protocol. The remaining cellular content was stained with anti-human CD45 (PacBlue or PECy7, clone HI30, BioLegend #304029 or #304016) and biotinylated cetuximab, followed by PE-conjugated streptavidin. All samples were analyzed on a MACSQuant VYB flow cytometer (Miltenyi), and the resulting data were analyzed using the FlowJo software (TreeStar).

In vivo studies. Six- to eight-week-old NOD/SCID/IL-2Rγ^{null} (NSG) mice were obtained from UCLA Department of Radiation and Oncology. The protocol was approved by UCLA Institutional Animal Care and Used Committee. Mice were injected with EGFP⁺ firefly luciferase (ffLuc)-expressing Raji lymphoma cells or CHLA-255 neuroblastoma cells by tail-vein injection, and subsequently treated with CAR-T cells or cells expressing EGFRt only (negative control) via tail-vein injection. Details of the dose and timing of tumor injection, T-cell injection, and tumor re-challenge are indicated in the text and figures. Tumor progression/regression was monitored with an IVIS Illumina III LT Imaging System (PerkinElmer). Blood samples were harvested via retro-orbital bleeding 3 days post T-cell injection and every 10-13 days thereafter. Mice were euthanized at the humane endpoint. Bone marrow, spleen and liver were collected after euthanasia. Tissues were ground and passed through a 100-μm filter followed by red-blood-cell lysis prior to flow-cytometry analysis.

Bulk RNA-seq for CAR-T cells cultured ex vivo. CD14⁻/CD25⁻/CD62L⁺ naïve/memory T cells (TN/M) were isolated, activated, retrovirally transduced as described above. On day 16 or 18 post activation, T cells were MACS-sorted with Dead Cell Removal kit (Miltenyi) to remove apoptotic population and enriched for EGFRt⁺ subpopulation.

RNA library preparation and sequencing was done as follows: mRNAs were isolated using NEBNext Poly(A) mRNA Magnetic Isolation Module (New England BioLabs). RNA-seq libraries were generated using NEBNext Ultra II Directional RNA Library Prep Kit (New England BioLabs) following manufacturer's protocol. Libraries were sequenced on the Illumina NovaSeq S1 platform at the High Throughput Sequencing core at UCLA Broad Stem Cell Research Center with 50-bp paired-end reads.

Fastq files from RNA-seq were quality-examined by FastQC (Linux, v0.11.8). Reads were processed by cutadapt (Linux, v1.18) to remove reads with low quality (quality score <33) and to trim adapters. Trimmed reads were mapped to hg38 genome by Tophat2. Fragments assigned to each gene were counted by featureCounts function in subread package (Linux, v1.6.3) with ensembl 38 gene sets as references. Genes without at least 8 reads mapped in at least one sample were considered below reliable detection limit and eliminated. Read counts were normalized by Trimmed Mean of M-values method (TMM normalization method in edgeR running on R v3.6.3) to yield FPKM (fragments per millions per kilobases) values.

Gene set enrichment analysis (GSEA), Enrichr analysis, and principal component analysis (PCA). ANOVA cluster genes were assigned using hierarchical clustering via the centroid method, with the cutHeight set at 2.8. GO analysis was performed using GSEA software (v4.1.0, Broad Institute). Expression values of differentially expressed genes were input to the program and using a curated list of 2493 T-cell-relevant gene sets selected from current Molecular Signatures Database (MSigDB; 2020 version) gene set, namely the Hallmark and Kyoto Encyclopedia of Genes and Genomes (KEGG; 2021 version) gene databases. Differentially expressed genes were used to perform KEGG (2021 version) and MSigDB Hallmark (2020 version) pathway enrichment using Enrichr. Top 3 pathways with adjusted p value that were less than 0.05 were chosen to display. Heatmaps for differentially expressed genes were generated using heatmap.plus, pheatmap and ggplot2 packages in R (version 3.6.3). Volcano plots and GSEA dot plots were generated using ggplot2 in R (version 3.6.3). PCA analysis was conducted using pcaExplorer (version 3.15).

Statistical Analysis. Statistical tests including two-tailed, unpaired, two-sample Student's t test with Sidak correction for multiple comparisons, log-rank Mentel-Cox test with Holm-Sidak correction for multiple comparisons, and Mann-Whitney U test with Holm-Sidak correction for multiple comparisons were performed using GraphPad Prism V8. One-way ANOVA test and pairwise differential gene expression analysis in RNA-seq was performed with glmQLFTest function in edgeR. Fisher's exact test and the Benjamini-Hochberg method were used to calculate p values and adjusted p values, respectively, in Enrichr.

### B. Results

### 1. CAR Expression Differentially Activates PI3K/AKT Signaling and Downstream T-Cell Functions

The panel of protein engineered CD20 CARs provides an intriguing window to examine the relationship between CAR tonic signaling and in vivo efficacy. We noted with interest that rituximab CAR-T cells exhibit strong T-cell activation in the absence of antigen stimulation, whereas Leu16 CAR-T cells appear completely quiescent at rest (data not shown). The inventors asked whether an intermediate level of antigen-independent activation may be key for a CAR-T cells' ability to productively respond to tumor challenge in vivo. If a causal relationship between tonic signaling and in vivo efficacy can be made, then one can in principle use tonic signaling as the primary design parameter in developing novel CARs, circumventing the need to carry out time- and resource-intensive in vivo experiments in determining functional efficacy.

To understand the molecular pathways underlying CD20 CAR tonic signaling, transcriptional analyses were performed on unstimulated CD20 CAR-T cells at the end of the CAR-T cell manufacturing cycleGene ontology (GO) and pathway analyses indicate unstimulated rituximab CAR-T cells significantly upregulate signaling pathways related to PI3K, phospholipase C (PLC), and mechanistic target of rapamycin complex 1 (mTORC1), as well as T-cell activation and proliferation (data not shown). Furthermore, unstimulated rituximab CAR-T cells exhibit an increase in metabolic processes and cytokine signaling relative to other CD20 CAR-T cells (data not shown). Together, these transcriptomic signatures indicate rituximab CAR signaling through the CD32 and CD28 domains despite lack of antigen stimulation. Although a number of genes were enriched in CD19, Leu16, and EGFRt-only samples, these genes did not correspond to any pathway with statistical significance.

The strong antigen-independent activation of rituximab CAR-T cells revealed by transcriptomic analysis was corroborated at the functional level with increased cell division, TNF-α production, activation-marker expression, and elevated metabolic flux, all in the absence of antigen stimulation (data not shown). In contrast, unstimulated Leu16 CAR-T cells were enriched in resting and memory T-cell phenotypes (data not shown), and exhibited minimal antigen-independent cell proliferation, cytokine production, activation-marker expression, and metabolic flux (data not shown). In fact, among all the CAR-T cell lines tested (including CD19 CAR-T cells), Leu16 CAR-T cells were the most similar to mock-transduced (EGFRt-only) T cells and exhibited a nearly complete lack of antigen-independent T-cell activation (data not shown). These results demonstrate that (a) CAR expression alone, without antigen stimulation, can drive divergent CAR-T cell phenotype, likely through the PI3K/AKT signaling axis; slight alterations in CAR sequences can lead to dramatic changes in CAR-T cell function; and strong antigen-independent activation may be detrimental to CAR-T cell function, but minimizing basal activation does not necessarily maximize anti-tumor-efficacy.

2. Pharmacological Inhibition of PI3K/AKT and mTOR Signaling Modestly Enhances Anti-Tumor Efficacy in Tonic Signaling Rituximab CAR-T Cells

The inventors sought to understand whether the intermediate level of antigen-independent signaling and strong antigen-dependent effector function are a mere coincidence, or if antigen-independent activation level is a parameter that one could productively tune to enhance CAR-T cell function. To do so, we explored the use of small-molecule inhibitors of signaling pathways identified through our transcriptomic analysis of unstimulated CAR-T cells. If proven effective, such a pharmacological modulation approach would be easily implementable during the manufacturing of clinical CAR-T cell products, without necessitating a change in the CAR protein construct.

To maximize clinical adaptability, we identified small-molecule inhibitors targeting PI3K, AKT, mTORC1, c-MYC, and SREBP that have undergone pre-clinical testing or have already been FDA-approved for human use (FIG. 1A). Rituximab CAR-T cells treated with PI3K8 (CAL-101) and AKT (AZD5363) inhibitors significantly reduced antigen-independent expression of activation and exhaustion markers, TNF-α production, and cell division compared to vehicle (DMSO)-treated rituximab CAR-T cells (FIG. 1B-D). In contrast, inhibition of c-Myc (MYCi975) and SREBP (betulin) increased most measures of antigen-independent CAR-T cell activation (FIG. 1B-D). Concordantly, PI3KI (CAL-101)- and AKT (AZD5363)-inhibited rituximab CAR-T cells preserved a greater fraction of naïve and stem-cell memory T (Tn/scm) cells compared to their vehicle-treated counterpart, whereas c-Myc (MYCi975) and SREBP inhibition (betulin) accelerated T-cell differentiation (FIG. 1E). Interestingly, mTORC1 inhibition by rapamycin led to reduced CD137 and PD-1 expression as well as enrichment of Tn/scm cells, but it had insignificant impact on CD69 expression while increasing TNF-α production in the absence of antigen stimulation (FIG. 1B-E), suggesting a branching of regulatory pathways controlling these different outputs upstream of mTOR signaling. Notably, mTORC1 (rapamycin) and SREBP (betulin) inhibition led to substantial decreases and increases in antigen-independent CAR-T cell metabolic activity, respectively (data not shown), indicating that modulating mTORC1 signaling or amplifying tonic signaling impacts antigen-independent metabolic activity. However, all pharmacologically inhibited rituximab CAR-T cells were unable to expand as effectively ex vivo (FIG. 1F), highlighting a limitation when using drug inhibitors.

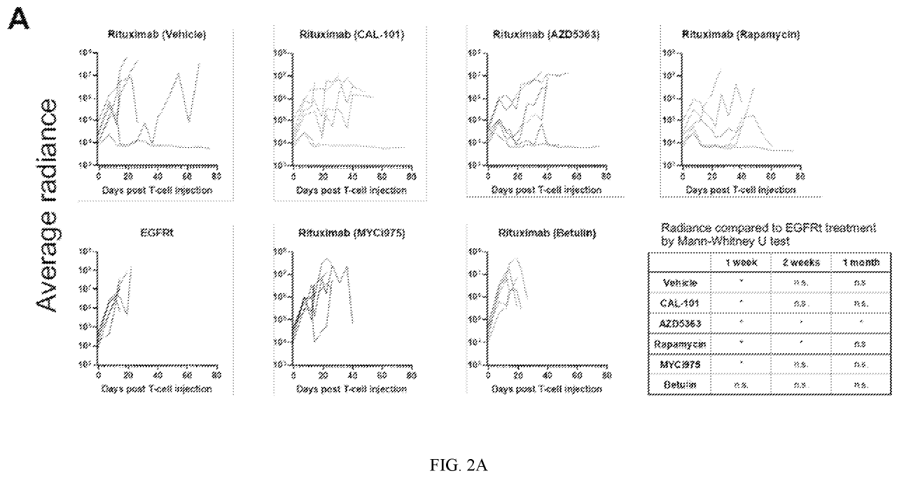

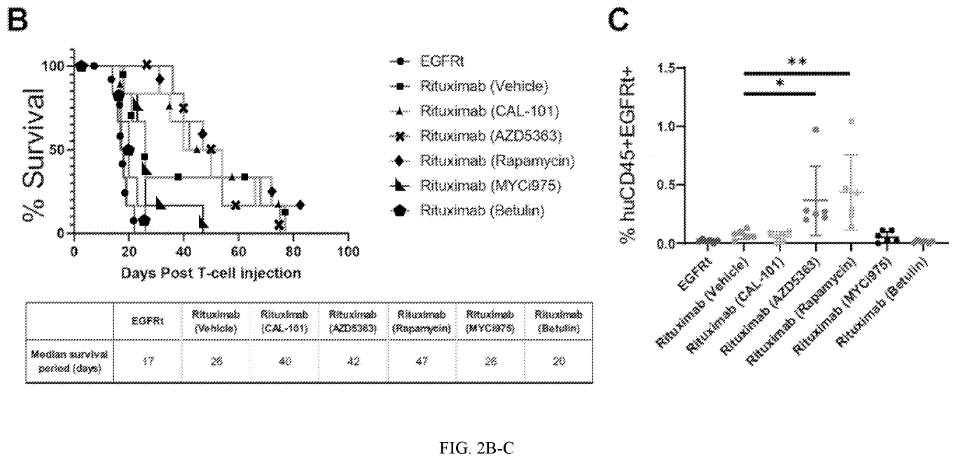

All inhibitor-treated rituximab CAR-T cells lysed Raji tumor cells as well as their vehicle-treated counterpart, indicating that ex vivo culture modulation did not impair CAR-T cell function (FIG. 1G). We next evaluated whether the change in antigen-independent T-cell activation induced by temporary pharmacological modulation during ex vivo T-cell manufacturing would translate to changes in in vivo anti-tumor efficacy. NSG mice bearing Raji tumor xenografts were treated with rituximab CAR-T cells preconditioned with various modulators (FIG. 1A). Since each group was treated with T cells expressing the same CAR, this comparison was not subject to confounding effects of CAR protein sequence variation. Results showed PI3Kδ (CAL-101)-, AKT (AZD5363)-, and mTORC1 (rapamycin)-preconditioned rituximab CAR-T cells delayed tumor outgrowth and prolonged median survival compared to vehicle (DMSO)-treated rituximab CAR-T cells by 1.5, 1.6, and 1.8 folds, respectively (FIG. 2A-B). Furthermore, AKT (AZD5363)- and mTORC1 (rapamycin)-inhibited rituximab CAR-T cells showed significantly increased in vivo expansion compared to the control (FIG. 1C), coinciding with their prolonged suppression of tumor outgrowth (FIG. 2A). In contrast, animals treated with c-Myc (MYCi975)- and SREBP (betulin)-preconditioned rituximab CAR-T cells, which exhibited increased antigen-independent activation in culture, resulted in accelerated tumor progression compared to the control group (FIG. 2A-B). These results support the concept that tuning the level of antigen-independent activity can indeed impact CAR-T cell function. Specifically, for a CAR that triggers strong basal T-cell activation, dampening antigen-independent activation enhances in vivo anti-tumor efficacy while further increasing activation diminishes in vivo function (FIG. 3A).

3. Pharmacological Inhibition of mTOR Signaling Modestly Decreases Tonic Signaling and Enhances Anti-Tumor Efficacy in Leu16 CAR-T Cells

The panel of PI3K/AKT pharmacological inhibitors allowed us to decrease or increase baseline antigen-independent activation of a strong tonic signaling CAR, providing evidence that high levels of tonic signaling causes CAR-T cell dysfunction, which can be rescued by minimization of tonic signaling (FIG. 3A). We wanted to further test our hypothesis that enhanced anti-tumor functionality could be achieved by an intermediate levels of tonic signaling. In order to do so, we reasoned that we could repurpose inhibitors of c-MYC (MYCi975) and SREBPs (betulin), which increased tonic signaling in rituximab CAR-T cells, to elevate Leu16 CAR-T cell tonic signaling (FIG. 3B). If the hypothesis were true, we would expect Leu16 CAR-T cell in vivo efficacy to improve, concomitant with minimal benefits towards tumor-killing efficacy with mTORC1 (rapamycin) inhibition (FIG. 3B).

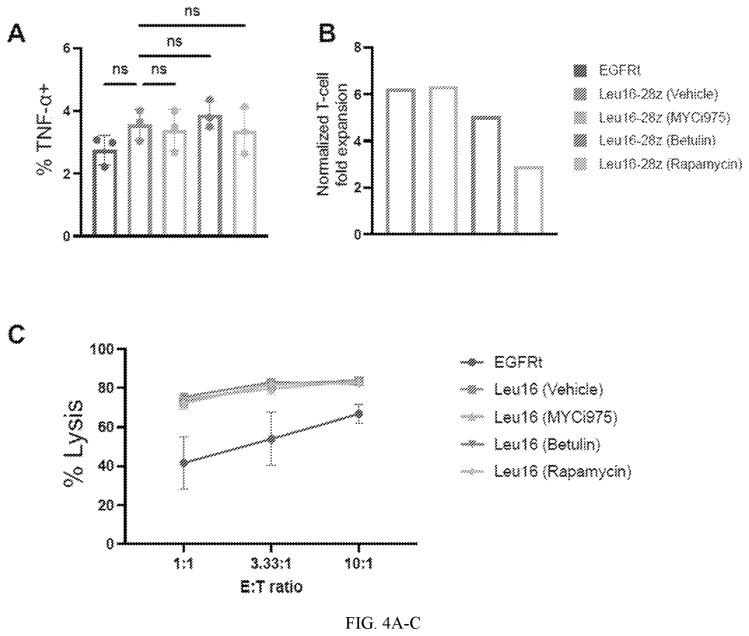

In contrast to what we observed in rituximab CAR-T cells, which have a high basal level of tonic signaling, pharmacological inhibition of Leu16 CAR-T cells targeting c-MYC (MYCi975), SREBPs (betulin), and mTORC1 (rapamycin) dampened tonic signaling Leu16 CAR-T cells, which have a very low basal level of tonic signaling, when treated at equivalent concentrations as shown in FIG. 1A. Inhibition by MYCi975, betulin, and rapamycin resulted in increased enrichment of Tn/scm cells relative to their vehicle counterpart (FIG. 3C), with rapamycin preserving the largest pool of Tn/scm cells. Similarly, all pharmacological inhibitors tested leds to significant decreases in activation and exhaustion marker expression (FIG. 3D). Furthermore, inhibition of mTORC1 by rapamycin significantly ablated antigen-independent proliferation in Leu16 CAR-T cells (FIG. 3E). It should be noted, however, that while the differences in T-cell subtype, exhaustion and activation marker expression, and antigen-independent proliferation were significant, the absolute differences between vehicle and drug-inhibited groups are rather small. This is likely due to the fact that Leu16 CAR-T cells tonic signal minimally, leaving a small dynamic range to be modulated. In fact, the pharmacologically induced modulation in tonic signaling led to no significant differences in TNF-α production (FIG. 4A). Despite modest changes in antigen-independent Leu16 CAR-T cell behavior, mTORC1-inhibited Leu16 CAR-T cells were unable to expand as effectively as its vehicle counterpart (FIG. 4B).

Pharmacologically modulated Leu16 CAR-T cells lysed Raji tumor cells equally well as well as their vehicle-treated counterpart, demonstrating no overt impairment of CAR-T cell function (FIG. 4C). We proceeded to evaluate whether the modest changes observed in Leu16 CAR tonic signaling would potentiate differences in anti-tumor responses in a Raji lymphoma animal model. To our surprise, we found that mTORC1 (rapamycin)-inhibited Leu16 CAR-T cells, which showed the strongest decrease in in vitro measures of tonic signaling, had superior in vivo functionality compared to its vehicle treated counterpart (FIG. 3F-G). In fact, SREBP (betulin)-inhibited Leu16 CAR-T cells, which also saw a significant decrease in tonic signaling, also translated to prolonged tumor control and survival (FIG. 3F-G). Taken together, these results suggest that reducing tonic signaling in an already minimally tonic signaling CAR can further augment anti-tumor efficacy, and that mTORC1 inhibition by rapamycin during ex vivo can be a generalizable strategy to augment CAR-T cell efficacy, irrespective of baseline tonic signaling levels.

### C. Discussion

Through transcriptomic as well as functional analyses, we observed that rituximab CAR-T cells exhibit strong T-cell activation in the absence of antigen stimulation, whereas Leu16 CAR-T cells appear completely quiescent at rest. The inventors hypothesized that intermediate levels of tonic signaling are beneficial for CAR-T cells. To test this, they needed test systems wherein minor differences in amino acid sequence would not serve as confounding factors. Transcriptomic analyses of CD20 CAR variants led us to hypothesize that PI3K/AKT signaling was the primary driver of CD20 CAR tonic signaling. Pharmacological modulation of PI3K/AKT signaling nodes using pre-clinically tested inhibitors confirmed this hypothesis. Furthermore, pharmacological tuning of antigen-independent CAR signaling is an attractive cell engineering strategy as it is straightforward to implement and obviates the need of rational protein design, a non-trivial task. We found that inhibitors targeting upstream signaling targets—namely PI3K, AKT, and mTORC1—dampened tonic signaling while enhancing in vivo efficacy. To our surprise, inhibitors targeting downstream signaling pathways, c-MYC and SREBP, further amplified tonic signaling in rituximab CAR-T cells, which worsened tumor-killing efficacy. This demonstrated a causal relationship between strong tonic signaling and worsened anti-tumor efficacy.

Inhibition of tonic signaling in rituximab CAR-T cells confirmed that minimizing tonic signaling was beneficial. However, to further test the hypothesis that intermediate levels of tonic signaling are beneficial, we sought to increase tonic signaling in the Leu16 CAR that tonic signals minimally to an intermediate tonic signaling state. To our surprise, c-MYC and SREBP inhibitors that increased rituximab CAR-T cell tonic signaling modestly decreased tonic signaling in Leu16 CAR-T cells, as did mTORC1 inhibition. Functionally, mTORC1-inhibited Leu16 CAR-T cells, which saw the largest decrease in tonic signaling, led to significant tumor-killing improvements compared to vehicle-treated Leu16 CAR-T cells. Although not statistically significant, SREBP-inhibited Leu16 CAR-T cells, which saw a reduction smaller reduction in tonic signaling compared to mTORC1 inhibition, also modestly improved tumor control and survival. Collectively, the Leu16 data does not support the hypothesis that maintaining intermediate levels of CAR tonic signaling increases CAR efficacy. Furthermore, the data show that the outcomes of pharmacological PI3K/AKT modulation is contingent on whether the CAR expressed has high or low levels of tonic signaling.

All of the methods disclosed and claimed herein can be made and executed without undue experimentation in light of the present disclosure. While the compositions and methods of this invention have been described in terms of preferred aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. The references cited in the disclosure, to the extent that they provide exemplary procedural or other details supplementary to those set forth herein, are specifically incorporated herein by reference.

What is claimed is:

1. A method for modifying a chimeric antigen receptor (CAR)-T cell comprising contacting the CAR-T cell ex vivo with an inhibitor, wherein the inhibitor comprises an inhibitor of PI3K, AKT, mTOR, c-MYC, SREBP, or combinations thereof.

2. A method for treating a subject, the method comprising administering an inhibitor of PI3K, AKT, mTOR, c-MYC, SREBP, or combinations thereof, wherein the subject is being treated with CAR-T cell therapy.

3. A method for treating a subject comprising administering an inhibitor of PI3K, AKT, mTOR, c-MYC, SREBP and a CAR-T cell therapy to the subject.

4. The method of claim 2 or 3, wherein the inhibitor and CAR-T cell therapy are administered at the same time.

5. The method of claim 2 or 3, wherein the inhibitor therapy is administered prior to the CAR-T cell therapy.

6. The method of claim 2 or 3, wherein the inhibitor therapy is administered after the CAR-T cell therapy.

7. The method of any one of claims 1-6, wherein the inhibitor is selected from CAL-101, AZD5363, Rapamycin, MYCi975, Betulin, and combinations thereof.

8. The method of claim 1 or 7, wherein the concentration of the inhibitor in contact with the cell is 1 nM-1 mM.

9. The method of any one of claim 1 or 7-8, wherein contacting the CAR-T cell ex vivo with the inhibitor comprises culturing the cell in cell medium comprising the inhibitor.

10. The method of claim 9, wherein the concentration of the inhibitor in the cell medium is 1 nm-1 mM.

11. The method of claim 10, wherein the inhibitor comprises CAL-101 at a concentration of 1 μM.

12. The method of claim 10, wherein the inhibitor comprises AZD5363 at a concentration of 1 μM.

13. The method of claim 10, wherein the inhibitor comprises Rapamycin at a concentration of 100 nM.

14. The method of claim 10, wherein the inhibitor comprises MYCi975 at a concentration of 2 μM.

15. The method of claim 10, wherein the inhibitor comprises Betulin at a concentration of 10 μM.

16. The method of any one of claims 9-15, wherein the cell medium further comprises cytokines.

17. The method of claim 16, wherein the cytokines comprise IL-2 and/or IL-15.

18. The method of claim 17, wherein the concentration of IL-2 is 50 U/mL and/or the concentration of IL-15 is 1 ng/mL.

19. The method of claim 17, wherein the concentration of IL-2 is 50 U/mL and/or the concentration of IL-15 is 0.5 ng/mL.

20. The method of any one of claims 9-18, wherein the cell medium excludes CD3 and/or CD28 activating molecules.

21. The method of any one of claims 1-20, wherein the CAR-T cell is derived from a CD8+ T cell.

22. The method of any one of claims 1-21, wherein the CAR-T cell is derived from a CD3+ T cell.

23. The method of any one of claims 1-20, wherein the CAR-T cell is derived from a naïve or memory T cell.

24. The method of claim 23, wherein the naïve or memory T cell comprises a CD62+ T cell.

25. The method of claim 24, wherein the cells have not been depleted for CD14 and CD25− positive cells and/or are not derived from a population of cells that are depleted for CD14 and CD25-positive cells.

26. The method of claim 23 or 24, wherein the naïve or memory T cell comprises a CD14−/CD25−/CD62+ T cell.

27. The method of any one of claims 1-26, wherein the CAR-T cell is contacted with the inhibitor for a period of time of at least 48 hours.

28. The method of any one of claims 1-27, wherein the CAR-T cell comprises stimulated CAR-T cells.

29. The method of claim 28, wherein the CAR-T cell has been stimulated with CD3 and/or CD28 antibodies or antigen-binding fragments.

30. The method of claim 29, wherein the CAR-T cell has been stimulated with CD3/CD28 Dynabeads.

31. The method of claim 29, wherein the CAR-T cell has been stimulated with TransAct.

32. The method of any one of claims 1-31, wherein the method further comprises determining the metabolic activity level of the cell.

33. The method of claim 32, wherein the metabolic activity is determined by mass spectrometry, liquid chromatography-mass spectrometry (LC-MS), metabolite extraction and analysis assays, an Agilent Seahorse XF ATP Real-Time rate assay, and/or direct measurement of metabolites.

34. The method of claim 32 or 33, wherein the cell is determined to have a high level of metabolic activity.

35. The method of claim 32 or 33, wherein the cell is determined to have a low level of metabolic activity.

36. The method of any one of claims 1-35, wherein the method is further defined as a method for increasing antigen-independent activation of the CAR-T cells, and wherein the inhibitor comprises a c-MYC and/or SREBP inhibitor.

37. The method of any one of claims 1-35, wherein the method is further defined as a method for decreasing antigen-independent activation of the CAR-T cells, and wherein the inhibitor comprises one or more of a PI3K, AKT, or mTOR inhibitor.

38. The method of any one of claim 1, or 7-37, wherein the cell has been isolated from a patient by leukapheresis.

39. The method of any one of claims 1-38, wherein the CAR comprises a CD19, CD20, bispecific, monospecific, IL13Ra2, BCMA, GD2, EGFRVIII, TGF-β, or CS1.

40. The method of claim 39, wherein the CAR comprises a CD20 CAR.

41. The method of claim 40, wherein the CD20 CAR comprises a Rituximab-based scFv.

42. The method of claim 41, wherein the CD20 CAR comprises a scFv comprising a variable heavy (VH) and variable light (VL) region, wherein the VH comprises complementarity determining regions (CDRS) HCDR1, HCDR2, and HCDR3 and the VL comprises LCDR1, LCDR2, and LCDR3; wherein HCDR1, HCDR2, HCDR3, LCDR1, LCDR2, and LCDR comprise the amino acid sequences of SEQ ID NOS: 19-24, respectively, or an amino acid sequence with at least 80% sequence identity to SEQ ID NOS: 19-24, respectively.

43. The method of claim 42, wherein the VL and VH comprise the amino acid sequences of SEQ ID NOS: 3-4, respectively, or an amino acid sequence with at least 80% sequence identity to SEQ ID NOS: 3-4, respectively.

44. The method of claim 40, wherein the CAR comprises a Leu16 scFv.

45. The method of claim 44, wherein the CD20 CAR comprises a scFv comprising a variable heavy (VH) and variable light (VL) region, wherein the VH comprises complementarity determining regions (CDRS) HCDR1, HCDR2, and HCDR3 and the VL comprises LCDR1, LCDR2, and LCDR3; wherein HCDR1, HCDR2, HCDR3, LCDR1, LCDR2, and LCDR comprise the amino acid sequences of SEQ ID NOS: 5-10, respectively, or an amino acid sequence with at least 80% sequence identity to SEQ ID NOS: 5-10, respectively.

46. The method of claim 44 or 45, wherein the VL and VH comprise the amino acid sequences of SEQ ID NOS: 1-2, respectively, or an amino acid sequence with at least 80% sequence identity to SEQ ID NOS: 1-2, respectively.

47. The method of any one of claims 1-46, wherein the cell has been transduced with a viral vector comprising a nucleic acid encoding a CAR.

48. The method of claim 47, wherein the viral vector comprises a lentiviral vector.

49. The method of any one of claims 1-45, wherein a nucleic acid encoding the CAR has been transferred into the cell by a non-viral method.

50. The method of any one of claims 1-49, wherein the method further comprises cryopreservation of the cell before or after contacting the cell with the inhibitor.

51. A CAR-T cell made by the method of any one of claim 1 or 7-50.

52. A population of CAR-T cells made by the method of any one of claim 1 or 7-50 or derived from the cell of claim 51.

53. The population of cells of claim 52, wherein the population comprises at least 5% CD62L+CAR−T+ cells.

54. The population of cells of claim 52 or 53, wherein the cells comprise at least 15% CD3+ cells.

55. The population of cells of any one of claims 52-54, wherein the cells have an average of 1-3 copies of the nucleic acid encoding the CAR per transduced cell.

56. The population of cells of any one of claims 52-55, wherein at least 70% of the cells are viable after thawing.

57. The population of cells of any one of claims 52-56, wherein the population comprises a mixture of CD4+ and CD8+ T cells.

58. The population of cells of claim 57, wherein at least 5% of the cells are CD4+.

59. The population of cells of any one of claims 52-58, wherein at least 15% of the cells are CD8+.

60. A method for treating a subject comprising administering the cell or cells of any one of claims 51-59.

61. The method of claim 60, wherein 1×10⁶-2×10⁸ cells are administered.

62. The method of claim 61, wherein the cells are determined to be positive for expression of the CAR.

63. The method of any one of claims 60-62, wherein the cells are autologous cells.

64. The method of any one of claims 60-63, wherein the subject is being treated with an additional therapy.

65. The method of any one of claims 60-64, wherein the method further comprises administering an inhibitor of PI3K, AKT, mTOR, c-MYC, SREBP, or combinations thereof to the subject.

66. The method of claim 65, wherein the inhibitor is administered at the same time of the cells.

67. The method of claim 65, wherein the inhibitor is administered before the cells.

68. The method of claim 65, wherein the inhibitor is administered after the cells.

69. The method of any one of claims 65-68, wherein the inhibitor is selected from CAL-101, AZD5363, Rapamycin, MYCi975, Betulin, and combinations thereof.

70. The method of any one of claims 60-69, wherein the method further comprises determining the metabolic activity level of the administered cell, of immune cells from the subject, and/or of cells from a biological sample from the subject.

71. The method of any one of claims 60-69, wherein the subject has had the metabolic activity level determined in immune cells and/or a cells from a biological sample from the subject.

72. The method of claim 70 or 71, wherein the biological sample comprises a biopsy or sample of cancer cells obtained from the subject.

73. The method of any one of claims 70-72, wherein the metabolic activity is determined by mass spectrometry, liquid chromatography-mass spectrometry (LC-MS), metabolite extraction and analysis assays, an Agilent Seahorse XF ATP Real-Time rate assay, and direct measurement of metabolites.

74. The method of any one of claims 70-73, wherein the cell is determined to have a high level of metabolic activity.

75. The method of any one of claims 70-73, wherein the cell is determined to have a low level of metabolic activity.

76. The method of any one of claims 71-75, wherein the immune cells from the subject have been determined to have a low level of metabolic activity.

77. The method of any one of claims 71-75, wherein the immune cells from the subject have been determined to have a high level of metabolic activity.

78. The method of any one of claims 71-77, wherein the cells from the biological sample from the subject have been determined to have a low level of metabolic activity.

79. The method of any one of claims 71-77, wherein the cells from the biological sample from the subject have been determined to have a high level of metabolic activity.

80. The method of any one of claims 60-79, wherein the method is further defined as a method for increasing antigen-independent activation of the CAR-T cells, and wherein the inhibitor comprises a c-MYC and/or SREBP inhibitor.

81. The method of any one of claims 65-79, wherein the method is further defined as a method for decreasing antigen-independent activation of the CAR-T cells, and wherein the inhibitor comprises one or more of a PI3K, AKT, or mTOR inhibitor.

82. The method of any one of claims 60-81, wherein the method further comprises administration of an additional therapy.

83. The method of claim 82, wherein the additional therapy comprises a chemotherapy.

84. The method of claim 82 or 83, wherein the additional therapy comprises lymphodepletion.

85. The method of any one of claims 82-84, wherein the additional therapy comprises fludarabine and/or cyclophosphamide.

86. The method of any one of claims 82-85, wherein the additional therapy is given prior to administration of the cells.

87. The method of claim 86, wherein the additional therapy is given to the subject starting at five days prior to administration of the cells.

88. The method of any one of claims 85-87, wherein the subject is administered 30 mg/m²/day for 30 min of fludarabine and 500 mg/m²/day for 60 min of cyclophosphamide for 3 days.

89. The method of any one of claims 85-87, wherein the method is for treating cancer and wherein the subject has cancer.

90. The method of claim 89, wherein the cancer is selected from lung cancer, prostate cancer, ovarian cancer, testicular cancer, brain cancer, neuroblastoma, sarcoma, osteocarcoma, skin cancer, melanoma, colon cancer, rectal cancer, gastric cancer, esophageal cancer, tracheal cancer, head & neck cancer, pancreatic cancer, liver cancer, breast cancer, ovarian cancer, glioblastoma, glioma, a B-cell malignancy, melanoma, leukemia, sarcomas of bone or soft tissue, cervical cancer, and vulvar cancer.

91. The method of claim 90, wherein the cancer comprises a B-cell malignancy.

92. The method of claim 91, wherein the B-cell malignancy comprises lymphoma or leukemia.

93. The method of claim 92, wherein the B-cell malignancy comprises non-Hodgkin B-cell lymphoma.

94. The method of claim 93, wherein the non-Hodgkin B-cell lymphoma is further classified as indolent non-Hodgkin lymphomas, follicular lymphoma, lymphoplasmacytic lymphoma, marginal zone lymphoma, nodal marginal zone lymphoma, gastric mucosa-associated lymphoid tissue (MALT) lymphoma, extragastric MALT lymphoma, mediterranean abdominal lymphoma, splenic marginal zone lymphoma, primary cutaneous anaplastic large cell lymphoma, diffuse large B-cell lymphoma, primary mediastinal B-cell lymphoma, follicular large cell lymphoma, anaplastic large cell lymphoma, cutaneous anaplastic large cell lymphoma, systemic anaplastic large cell lymphoma, extranodal NK-/T-cell lymphoma, lymphomatoid granulomatosis, angioimmunoblastic T-cell lymphoma, peripheral T-cell lymphoma, hepatosplenic T-cell lymphoma, subcutaneous panniculitis-like T-cell lymphoma, enteropathy-type intestinal T-cell lymphoma, intravascular large B-cell lymphoma, Burkitt lymphoma, lymphoblastic lymphoma, adult T-cell leukemia/lymphoma, mantle cell lymphoma, posttransplantation lymphoproliferative disorder, true histiocytic lymphoma, primary effusion lymphoma, or plasmablastic lymphoma.

95. The method of claim 92, wherein the B-cell malignancy comprises leukemia, and wherein the leukemia is further classified as chronic lymphocytic leukemia, small-lymphocytic leukemia, acute lymphocytic leukemia, acute myeloid leukemia, chronic myeloid leukemia, chronic myelomonocytic leukemia, pediatric leukemia, precursor B acute lymphoblastic leukemia, precursor T acute lymphoblastic leukemia, Burkitt's leukemia, acute biphenotypic leukemia, B-cell prolymphocytic leukemia, acute promyelocytic leukemia, acute myeloblastic leukemia, acute megakaryoblastic leukemia, hairy cell leukemia, T-cell prolymphocytic leukemia, large granular lymphocytic leukemia, adult T-cell leukemia, or clonal eosinophilias.

96. The method of claim 90, wherein the cancer comprises glioma.

97. The method of claim 96, wherein the glioma is selected from glioblastoma, malignant glioma, diffuse midline glioma, and diffuse intrinsic pontine glioma.

98. A composition comprising an inhibitor and CAR-T cells, wherein the inhibitor comprises an inhibitor of PI3K, AKT, mTOR, c-MYC, SREBP, or combinations thereof.

99. The composition of claim 98, wherein the composition further comprises cell medium.

100. The composition of claim 98 or 99, wherein the composition further comprises IL-2 and/or IL-15.

101. The composition of claim 100, wherein the concentration of IL-2 is 50 U/mL and/or the concentration of IL-15 is 1 ng/mL.

102. The composition of any one of claims 98-101, wherein the concentration of the inhibitor in the composition is 1 nm-1 mM.

103. The composition of claim 102, wherein the inhibitor comprises CAL-101 at a concentration of 1 μM.

104. The composition of claim 102, wherein the inhibitor comprises AZD5363 at a concentration of 1 μM.

105. The composition of claim 102, wherein the inhibitor comprises Rapamycin at a concentration of 100 nM.

106. The composition of claim 102, wherein the inhibitor comprises MYCi975 at a concentration of 2 μM.

107. The composition of claim 102, wherein the inhibitor comprises Betulin at a concentration of 10 μM.


SEQUENCE LISTING (51 sequences)

SEQ ID NO: 1 - protein, 106 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 2 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 3 - protein, 106 aa, synthetic construct [sequence omitted]

SEQ ID NO: 4 - protein, 121 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSS

SEQ ID NO: 5 - protein, 5 aa, synthetic construct
SYNMH

SEQ ID NO: 6 - protein, 17 aa, synthetic construct
AIYPGNGDTSYNQKFKG

SEQ ID NO: 7 - protein, 13 aa, synthetic construct
SNYYGSSYWFFDV

SEQ ID NO: 8 - protein, 10 aa, synthetic construct
RASSSVNYMD

SEQ ID NO: 9 - protein, 7 aa, synthetic construct
ATSNLAS

SEQ ID NO: 10 - protein, 9 aa, synthetic construct
QQWSFNPPT

SEQ ID NO: 11 - protein, 30 aa, synthetic construct
EVQLQQSGAELVKPGASVKMSCKASGYTFT

SEQ ID NO: 12 - protein, 14 aa, synthetic construct
WVKQTPGQGLEWIG

SEQ ID NO: 13 - protein, 32 aa, synthetic construct
KATLTADKSSSTAYMQLSSLTSEDSADYYCAR

SEQ ID NO: 14 - protein, 11 aa, synthetic construct
WGAGTTVTVSS

SEQ ID NO: 15 - protein, 23 aa, synthetic construct
DIVLTQSPAILSASPGEKVTMTC

SEQ ID NO: 16 - protein, 15 aa, synthetic construct
WYQKKPGSSPKPWIY

SEQ ID NO: 17 - protein, 32 aa, synthetic construct
GVPARFSGSGSGTSYSLTISRVEAEDAATYYC

SEQ ID NO: 18 - protein, 10 aa, synthetic construct
FGGGTKLEIK

SEQ ID NO: 19 - protein, 5 aa, synthetic construct
SYNMH

SEQ ID NO: 20 - protein, 17 aa, synthetic construct
AIYPGNGDTSYNQKFKG

SEQ ID NO: 21 - protein, 12 aa, synthetic construct
STYYGGDWYFNV

SEQ ID NO: 22 - protein, 10 aa, synthetic construct
RASSSVSYIH

SEQ ID NO: 23 - protein, 7 aa, synthetic construct
ATSNLAS

SEQ ID NO: 24 - protein, 9 aa, synthetic construct
QQWTSNPPT

SEQ ID NO: 25 - protein, 30 aa, synthetic construct
QVQLQQPGAELVKPGASVKMSCKASGYTFT

SEQ ID NO: 26 - protein, 14 aa, synthetic construct
WVKQTPGRGLEWIG

SEQ ID NO: 27 - protein, 32 aa, synthetic construct
KATLTADKSSSTAYMQLSSLTSEDSAVYYCAR

SEQ ID NO: 28 - protein, 11 aa, synthetic construct
WGAGTTVTVSS

SEQ ID NO: 29 - protein, 23 aa, synthetic construct
QIVLSQSPAILSASPGEKVTMTC

SEQ ID NO: 30 - protein, 15 aa, synthetic construct
WFQQKPGSSPKPWIY

SEQ ID NO: 31 - protein, 32 aa, synthetic construct
GVPVRFSGSGSGTSYSLTISRVEAEDAATYYC

SEQ ID NO: 32 - protein, 10 aa, synthetic construct
FGGGTKLEIK

SEQ ID NO: 33 - protein, 20 aa, synthetic construct
METDTLLLWVLLLWVPGSTG

SEQ ID NO: 34 - protein, 18 aa, synthetic construct
GSTSGGGSGGGSGGGGSS

SEQ ID NO: 35 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 28 aa, synthetic construct
MFWVLVVVGGVLACYSLLVTVAFIIFWV

SEQ ID NO: 37 - protein, 41 aa, synthetic construct
RSKRSRGGHSDYMNMTPRRPGPTRKHYQPYAPPRDFAAYRS

SEQ ID NO: 38 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 24 aa, synthetic construct
LEGGGEGRGSLLTCGDVEENPGPR

SEQ ID NO: 40 - protein, 255 aa, synthetic construct
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQA

SEQ ID NO: 41 - protein, 9 aa, synthetic construct
YPYDVPDYA

SEQ ID NO: 42 - protein, 8 aa, synthetic construct
DYKDDDDK

SEQ ID NO: 43 - protein, 10 aa, synthetic construct
EQKLISEEDL

SEQ ID NO: 44 - protein, 5 aa, synthetic construct
GSGGS

SEQ ID NO: 45 - protein, 4 aa, synthetic construct
GGGS

SEQ ID NO: 46 - protein, 4 aa, synthetic construct
GGSG

SEQ ID NO: 47 - protein, 5 aa, synthetic construct
GGSGG

SEQ ID NO: 48 - protein, 5 aa, synthetic construct
GSGSG

SEQ ID NO: 49 - protein, 5 aa, synthetic construct
GSGGG

SEQ ID NO: 50 - protein, 5 aa, synthetic construct
GGGSG

SEQ ID NO: 51 - protein, 5 aa, synthetic construct
GSSSG